(19) United States
(12) Patent Application Publication
(10) Pub. No.: US 2017/0088597 A1
(43) Pub. Date: Mar. 30, 2017

(54) INTERLEUKIN-15 SUPERAGONIST SIGNIFICANTLY ENHANCES GRAFT-VERSUS-TUMOR ACTIVITY

(71) Applicants: Altor Bioscience Corporation, Miramar, FL (US); Thomas Jefferson University, Philadelphia, PA (US)

(72) Inventors: Hing C. Wong, Weston, FL (US); Emily K. Jeng, Miramar, FL (US); S. Onder Alpdogan, Philadelphia, PA (US)

(21) Appl. No.: 15/273,836

(22) Filed: Sep. 23, 2016

Related U.S. Application Data
(60) Provisional application No. 62/232,515, filed on Sep. 25, 2015.

(51) Int. Cl. C07K 14/54; C07K 14/715; A61K 35/17; A61K 35/28; A61K 38/20; A61K 38/17

(52) U.S. Cl. CPC C07K 14/5443; A61K 38/2086; A61K 38/1793; A61K 35/17; A61K 35/28; C07K 14/7155; C07K 2319/30; C07K 2319/32

(57) ABSTRACT

The invention features therapies using an IL-15-based superagonist complex to effectively treat subjects with cancer.

### CROSS-REFERENCE TO RELATED APPLICATIONS

This application claims the benefit of priority under 35 U.S.C. §119(e) to U.S. Provisional Application No. 62/232,515, filed on Sep. 25, 2015, which is incorporated herein by reference in its entirety.

### FIELD OF THE INVENTION

This invention relates generally to the field of therapies for treatment of cancer.

### BACKGROUND OF THE INVENTION

Prior to the invention described herein, there was a pressing need to develop new strategies to augment and/or direct immune activity against cancer cells.

### SUMMARY OF THE INVENTION

The invention is based, at least in part, on the surprising discovery that ALT-803, a complex of an interleukin-15 (IL-15) superagonist mutant and a dimeric IL-15 receptor α/Fc fusion protein, i.e., an IL-15N72D:IL-15RαSu/Fc complex (ALT-803), enhanced graft-versus-tumor (GVT) activity against hematologic malignancies in hematopoietic stem cell transplantation and donor leukocyte infusion (DLI) models without increasing graft-versus-host disease.

Thus, described herein is a method for treating a neoplasia in a subject. In one aspect, a subject is identified as having or at risk of developing a neoplasia. An effective amount of an adoptive cell therapy and an effective amount of a pharmaceutical composition comprising an IL-15:IL-15Rα complex is administered to the subject, thereby treating the neoplasia.

In certain embodiments, the soluble fusion protein complexes of the invention include an IL-15 polypeptide, IL-15 variant, or a functional fragment thereof and a soluble IL-15Rα polypeptide or a functional fragment thereof. In some cases, one or both of the IL-15 and IL-15Rα polypeptides further include an immunoglobulin Fc domain or a functional fragment thereof.

For example, the IL-15/IL-15Rα complex is an IL-15N72D:IL-15RαSu/Fc complex (ALT-803), wherein the ALT-803 comprises a dimeric IL-15RαSu/Fc and two IL-15N72D molecules. An exemplary IL-15N72D molecule comprises SEQ ID NO: 3. In some cases, the IL-15RαSu/Fc comprises SEQ ID NO: 6.

The subject is preferably a mammal in need of such treatment, e.g., a subject that has been diagnosed with a neoplasia or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Suitable neoplasias for treatment with the methods described herein include hematological cancer, chronic myelogenous leukemia, acute myelogenous leukemia, acute lymphoblastic leukemia, myelodysplasia, multiple myeloma, mantle cell lymphoma, B cell non-Hodgkin lymphoma, Hodgkin's lymphoma, chronic lymphocytic leukemia, B-cell neoplasms, B-cell lymphoma, leukemia, cutaneous T-cell lymphoma, T-cell lymphoma, a solid tumor, urothelial/bladder carcinoma, melanoma, lung cancer, renal cell carcinoma, breast cancer, gastric and esophageal cancer, head and neck cancer, prostate cancer, colorectal cancer, ovarian cancer, non-small cell lung carcinoma, sarcoma, mastocytoma and adenocarcinoma.

Preferably, administration of the compositions described herein also prevents future recurrence of neoplasia after treatment of the disease.

Exemplary effective doses of ALT-803 include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

Suitable effective doses of adoptive cell therapy include between 1×10³ and 1×10¹² cells/dose, e.g., 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, or 1×10¹² cells/dose. An exemplary effective dose of adoptive cell therapy is 5×10⁶ cells/dose.

In some cases, the ALT-803 (and/or adoptive cell therapy) is administered daily, e.g., every 24 hours. Or, the ALT-803 (and/or adoptive cell therapy) is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Alternatively, the ALT-803 (and/or adoptive cell therapy) is administered about once per week, e.g., about once every 7 days. Or, the ALT-803 (and/or adoptive cell therapy) is administered twice per week, three times per week, four times per week, five times per week, six times per week, or seven times per week. Exemplary effective weekly doses of ALT-803 include between 0.0001 mg/kg and 4 mg/kg body weight, e.g., 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. For example, an effective weekly dose of ALT-803 is between 0.1 μg/kg body weight and 400 μg/kg body weight. Alternatively, ALT-803 is administered at a fixed dose or based on body surface area (i.e., per m²). In some cases, subjects receive two 6-week cycles consisting of 4 weekly ALT-803 intravenous doses followed by a 2-week rest period. Ultimately, the attending physician or veterinarian decides the appropriate amount and dosage regimen.

The compositions described herein are administered systemically, intravenously, subcutaneous, intramuscularly, intravesically, or by instillation. The compositions, i.e., ALT-803 and adoptive cell therapy, may be administered simultaneously or sequentially.

Preferably, ALT-803 is administered in conjunction with adoptive cell therapies or transplants. Such adoptive cell therapies include, but are not limited to, allogeneic and autologous hematopoietic stem cell transplantation, donor leukocyte (or lymphocyte) infusion (DLI), adoptive transfer of tumor infiltrating lymphocytes, or adoptive transfer of T cells or NK cells. Optionally, the T cells or NK cells are engineered to express a suicide gene (e.g., an exogenous suicide gene), a chimeric antigen receptor gene, or a T cell receptor (TCR), e.g., a TCR specific to tumor antigens, or other genes to facilitate cell proliferation, survival, persistence, or activity against the tumor. The transferred cells could be obtained from various sources including the recipient (autologous) or related or unrelated donors. For example, adoptive cell therapy comprises transfer of allogeneic, autologous, syngeneic, related, unrelated, MHC-matched, MHC-mismatched, or haploidentical cells. Combination therapy with ALT-803 could be done in vivo, ex vivo, or in vitro, or combinations thereof.

Preferably, the ALT-803 stimulates proliferation or activation of adoptively transferred cells. For example, ALT-803 increases the number of adoptively transferred CD8⁺ T cells or NK cells in the subject. In another example, ALT-803 increases expression of IFN-γ, TNF-α, NKG2D, or CD107a in the adoptively transferred cells.

Preferably, the administration of ALT-803 and adoptive cell therapy increases graft-verse-tumor activity, but does not increase graft-verse-host disease. For example, the administration of ALT-803 and adoptive cell therapy results in a decreased number of tumor cells. In another example, the administration of ALT-803 and adoptive cell therapy results in a decrease in progression or a decrease in relapse of the neoplasia. Preferably, the administration of ALT-803 and adoptive cell therapy results in prolonged survival of the subject, e.g., a human subject, compared to an untreated subject. In some cases, the subject has a neoplasia that has relapsed from or is refractory to therapy administered previously.

Also provided are methods for treating a subject with a neoplasia that has relapsed from previous therapy, the method comprising administering to the subject an effective amount of a donor lymphocyte infusion therapy and an effective amount of ALT-803, thereby treating the neoplasia that has relapsed from previous therapy. Preferably, the method does not induce graft-verse-host disease. In some cases, the methods further comprise identifying a subject with a neoplasia who has relapsed from previously-administered therapy.

Also provided is a kit for the treatment of a neoplasia, the kit comprising an effective amount of ALT-803, an adoptive cell therapy, and directions for the use of the kit for the treatment of a neoplasia. For example, the adoptive cell therapy comprises hematopoietic stem cells, donor leukocytes, T cells, or NK cells. Alternatively, the kit comprises an effective amount of ALT-803 and an anti-neoplasia therapeutic such as an antibody, e.g., a tumor-specific antibody, or a chemotherapeutic agent, e.g., an alkylating agent (e.g., platinum-based drugs, tetrazines, aziridines, nitrosoureas, nitrogen mustards), an anti-metabolite (e.g., anti-folates, fluoropyrimidines, deoxynucleoside analogues, thiopurines), an anti-microtubule agent (e.g., vinca alkaloids, taxanes), a topoisomerase inhibitor (e.g., topoisomerase I and II inhibitors), a cytotoxic antibiotic (e.g., anthracyclines), a protein kinase inhibitor (e.g., tyrosine kinase inhibitors), or an immunomodulatory drug (e.g., thalidomide and analogs).

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound. An exemplary therapeutic agent is ALT-803.

By “ALT-803” is meant a complex comprising IL-15N72D noncovalently associated with a dimeric IL-15RαSu/Fc fusion protein and having immune stimulating activity. This complex is also referred to as IL-15 SA. In one embodiment, the IL-15N72D and/or IL-15RαSu/Fc fusion protein comprises one, two, three, four or more amino acid variations relative to a reference sequence. An exemplary IL-15N72D amino acid sequence is provided below.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The invention includes antibodies or fragments of such antibodies, so long as they exhibit the desired biological activity. Also included in the invention are chimeric antibodies, such as humanized antibodies. Generally, a humanized antibody has one or more amino acid residues introduced into it from a source that is non-human. Humanization can be performed, for example, using methods described in the art, by substituting at least a portion of a rodent complementarity-determining region for the corresponding regions of a human antibody.

The term “antibody” or “immunoglobulin” is intended to encompass both polyclonal and monoclonal antibodies. The preferred antibody is a monoclonal antibody reactive with the antigen. The term “antibody” is also intended to encompass mixtures of more than one antibody reactive with the antigen (e.g., a cocktail of different types of monoclonal antibodies reactive with the antigen). The term “antibody” is further intended to encompass whole antibodies, biologically functional fragments thereof, single-chain antibodies, and genetically altered antibodies such as chimeric antibodies comprising portions from more than one species, bifunctional antibodies, antibody conjugates, humanized and human antibodies. Biologically functional antibody fragments, which can also be used, are those peptide fragments derived from an antibody that are sufficient for binding to the antigen. “Antibody” as used herein is meant to include the entire antibody as well as any antibody fragments (e.g. F(ab′)2, Fab′, Fab, Fv) capable of binding the epitope, antigen or antigenic fragment of interest.

By “binding to” a molecule is meant having a physicochemical affinity for that molecule.

“Detect” refers to identifying the presence, absence or amount of the analyte to be detected.

By “disease” is meant any condition or disorder that damages or interferes with the normal function of a cell, tissue, or organ. Examples of diseases include neoplasias and infections.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

As used herein, the term “IL-15:IL-15Rα fusion protein complex” is a complex having IL-15 non-covalently or covalently bound to IL-15Rα. IL-15Rα can be either soluble or membrane bound. In some embodiments, IL-15Rα is the soluble domain of the native IL-15Rα polypeptide. The soluble IL-15Rα can be the IL-15Rα sushi domain or IL-15RαΔE3. In some cases, the soluble IL-15Rα is covalently linked to a biologically active polypeptide and/or to an IgG Fc domain. The IL-15 can be either IL-15 or IL-15 covalently linked to a second biologically active polypeptide. In some cases, IL-15 is covalently bound to the IL-15Rα domain via a linker. The IL-15 can also represent an IL-15 variant comprises one, two, three, four or more amino acid variations relative to a reference sequence. In one embodiment the IL-15 is IL-15N72D. In another embodiment, the IL-15:IL-15Rα fusion protein complex is ALT-803.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “neoplasia” is meant a disease or disorder characterized by excess proliferation or reduced apoptosis. Illustrative neoplasms for which the invention can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, gastric and esophageal cancer, head and neck cancer, rectal cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In particular embodiments, the neoplasia is multiple myeloma, beta-cell lymphoma, urothelial/bladder carcinoma or melanoma. As used herein, “obtaining” as in “obtaining an agent” includes synthesizing, purchasing, or otherwise acquiring the agent.

By “reduces” is meant a negative alteration of at least 5%, 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but are not limited to, a human or non-human mammal, such as a bovine, equine, canine, ovine, or feline. The subject is preferably a mammal in need of such treatment, e.g., a subject that has been diagnosed with B cell lymphoma or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

Treatment of patients with neoplasia may include any of the following: Adjuvant therapy (also called adjunct therapy or adjunctive therapy) to destroy residual tumor cells that may be present after the known tumor is removed by the initial therapy (e.g. surgery), thereby preventing possible cancer reoccurrence; neoadjuvant therapy given prior to the surgical procedure to shrink the cancer; induction therapy to cause a remission, typically for acute leukemia; consolidation therapy (also called intensification therapy) given once a remission is achieved to sustain the remission; maintenance therapy given in lower or less frequent doses to assist in prolonging a remission; first line therapy (also called standard therapy); second (or 3rd, 4th, etc.) line therapy (also called salvage therapy) is given if a disease has not responded or reoccurred after first line therapy; and palliative therapy (also called supportive therapy) to address symptom management without expecting to significantly reduce the cancer.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A, FIG. 1B, and FIG. 1C are bar charts showing that ALT-803 administration increases CD8⁺ T and NK cell numbers after transplantation. In FIG. 1A, lethally irradiated (11Gy) Balb/c recipients were transplanted with 5×10⁶ T cell depleted (TCD) bone marrow (BM) cells from B6 mice. ALT-803 was administered via IP injection at 1 μg per mouse in two doses on days 17 and 24. Mice were sacrificed on day 28 after transplant, and spleens, thymi and BM were harvested. Single cell suspensions were prepared and stained with anti-H2Kd, -CD3, -CD4, -CD8, -Gr-1, -NK1.1, and -B220 antibodies, and analyzed with a flow cytometer. Each group contains 5 mice. Splenic numbers of CD4⁺ T, CD8⁺ T, and NK cells are shown. *: P<0.05. In FIG. 1B and FIG. 1C, lethally irradiated (12Gy) CB6F1 recipients were transplanted with 5×10⁶ T-cell depleted (TCD) bone marrow (BM) cells from B6CBA mice. IL-15 super agonist was administered via IP injection at 1 μg per mouse in two doses on days 17 and 24. Mice were sacrificed at day 28 after transplant, and spleens, thymi and BM were harvested. After preparation of single cell suspensions, cells were stained with anti-H2Kd, -CD4, -CD8 (FIG. 1B). Some splenocytes were also incubated as described for intracellular staining, then harvested and stained with anti-H2Kd, -CD4, -CD8 and IFN-γ antibodies and analyzed by flow cytometery (FIG. 1C). Each group contains 5 mice. *: P<0.05

FIG. 2A and FIG. 2B show that ALT-803 administration increases CD8⁺ CD44⁺ and CD8⁺NKG2D⁺ effector/memory T cells. Lethally irradiated (11Gy) CB6F1 recipients were transplanted with 5×10⁶ T cell depleted (TCD) bone marrow (BM) cells from B6 mice. ALT-803 was administered via IP injection at 1 μg per mouse on days 28, 35 and 42. Mice were sacrificed on day 49 after transplant, and spleens were harvested. Single cell suspensions were prepared and stained with anti-H2Kd, -CD3, -CD4, -CD8, -CD44 and -NKG2D antibodies. Cells were acquired and analyzed by flow cytometery (FIG. 2A and FIG. 2B).

FIG. 3A and FIG. 3B show that ALT-803 administration increases cytokine secretion and proliferation of CD8⁺ T cells in recipients of CFSE labeled T cells. Lethally irradiated (1300 cGy) either B6D2F1 (FIG. 3A) or B6 (Ly5.1) mice (FIG. 3B) were transplanted with CFSE labeled B6 splenocytes (30×10⁶) on day 0 and given either ALT-803 or a vehicle control (Day 0 post infusion). Mice were sacrificed on day 3 after CFSE labeled leukocyte infusion and splenocytes were stained with anti-CD4, -CD8, CD45.1 and -H2Kd antibodies. Cells were then analyzed by flow cytometry. Intracellular staining with anti-IFN-γ and anti-TNF-α antibodies after PMA and ionomycin stimulation was performed. Red line indicates boundary of isotypic control and arrows indicate increase in IFN-γ secretion in the slow proliferating CD8⁺ T cells.

FIG. 4A, FIG. 4B, and FIG. 4C show that ALT-803 administration increases GVT activity after transplant. Lethally irradiated (13Gy) B6D2F1 recipients were transplanted with 5×10⁶ T cell depleted (TCD) bone marrow (BM) cells from B6 mice. All recipients also received 1×10⁴ P815 cells on the day of transplantation along with 5×10⁴ (FIG. 4A) or 1×10⁵ (FIG. 4B) purified B6 T cells. ALT-803 was administered via IP injection at 2.5 μg per mouse in two doses on days 7 and 14. Kaplan Mayer curves for this transplant modality are depicted as follows; vehicle control (black line) and IL-15 super-agonist (red line). *=p<0.05, and each group had 15 mice. In FIG. 4C, lethally irradiated (13Gy) CB6F1 recipients were transplanted with 5×10⁶ T cell depleted (TCD) bone marrow (BM) cells from B6 mice. All recipients also received 5×10⁵ A20 cells on the day of transplantation along with 1×10⁵ purified B6 T cells. ALT-803 was administered via IP injection at 2.5 μg per mouse in two doses on days 7 and 14. Kaplan Mayer curves for this transplant modality are depicted as follows; vehicle control (black line) and IL-15 super-agonist (red line). *=p<0.05, and each group had 10 mice.

FIG. 5A, FIG. 5B, and FIG. 5C show that ALT-803 delays A20 lymphoma cells growth in recipients of HSCT. Lethally irradiated (12Gy) CB6F1 recipients were transplanted with 5×10⁶ T-cell depleted (TCD) bone marrow (BM) cells from B6 mice. All recipients also received 5×10⁵ A20 cells on the day of transplantation along with T cell infusion. ALT-803 was administered via IP injection at 2.5 μg per mouse in two doses on days 7 and 14. In vivo luminescent imaging is shown in FIG. 5A. Mice were injected with luciferin at 3.75 mg per mouse, allowed to incubate for 8 mins, and then imaged for 3 mins. Control group on the left, ALT-803 on the right. Photon intensity is calculated and shown in FIG. 5B. *=p<0.05, and each group had 10 mice.

FIG. 6A, FIG. 6B, FIG. 6C, and FIG. 6D show that ALT-803 increases anti-tumor activity after DLI in murine leukemia/lymphoma model. Lethally irradiated (12Gy) CB6F1 recipients were transplanted with 5×10⁶ T cell depleted (TCD) bone marrow (BM) cells from B6 mice. All recipients also received 5×10⁵ A20-TGL (H2K^{d}) lymphoma cells with triple fusion gene carrying luciferase activity on the day of transplantation. The recipients of transplant received none or 2.5×10⁵ B6 T cells isolated via CD5⁺ magnetic separation on day 14 after transplant. Animals received either IP injections of ALT-803 (1 μg per mouse) or control on days 17 and 24 after transplant. Survival and weight curves of the groups are depicted as FIG. 6A and FIG. 6B (N=10-20). Serial bioluminescence images were obtained by IVIS machine in varying time points as show. A20-TGL cells express luciferase protein allowing in vivo bioluminescent imaging; mice were injected with 3.75 mg Luciferin, incubated for 5 mins, and imaged for 3 mins. Serial bioluminescence images were obtained by IVIS machine in varying time points as show (FIG. 6C representative of independent of two experiments). Control group on the left, ALT-803 group on the right. Total flux (photons/sec) was measured for each mouse at each time point and plotted as a curve (FIG. 6D).

### DETAILED DESCRIPTION

The invention is based, at least in part, on the surprising discovery that ALT-803, a complex of an interleukin-15 (IL-15) superagonist mutant (IL-15N72D) and a dimeric IL-15 receptor α/Fc fusion protein, i.e., an IL-15N72D:IL-15RαSu/Fc complex (ALT-803), enhanced graft-versus-tumor (GVT) activity against hematologic malignancies in hematopoietic stem cell transplantation and donor leukocyte infusion (DLI) models without increasing graft-versus-host disease.

Allogeneic, and in some cases autologous, hematopoietic stem cell transplantation (HSCT) is the treatment of choice for many malignant hematological disorders (for reviews of HSCT and adoptive cell therapy approaches, see, Rager & Porter, Ther Adv Hematol (2011) 2(6) 409-428; Roddie & Peggs, Expert Opin. Biol. Ther. (2011) 11(4):473-487; Wang et al. Int. J. Cancer: (2015)136, 1751-1768; and Chang, Y. J. and X. J. Huang, Blood Rev, 2013. 27(1): 55-62). The efficacy of allogeneic HSCT as a curative option for hematological malignancy is influenced by a number of factors including the underlying disease, the pre-transplant conditioning regimen and the graft-versus-tumor (GVT) effect mediated by donor leukocytes within the graft. The last two factors must be balanced against transplant-related mortality (TRM). For example, reduced intensity conditioning regimens are now being used to provide sufficient immunosuppression for donor cell engraftment without the highly toxic, inflammatory cytokine storm′ induced by conventional myeloablative conditioning. These less toxic strategies permit use of HSCT in a population of previously ineligible patients. However, with the improvements to reduce non-relapse-related mortality rates, relapse has become the commonest cause of treatment failure. Infection and graft-versus-host disease are also major complications of allogeneic HSCT. Thus, additional therapeutic approaches are needed both to improve the clinical efficacy of HSCT without increased toxicities and to treat disease that has relapsed following HSCT.

Prior to the invention described herein, the strategies for treating relapse after allogeneic HSCT include withdrawal of immune suppression, a second allogeneic transplantation, or DLI. Withdrawal of immune suppression has been successful in limited case studies, although is rarely effective when used alone. Second allogeneic transplantation remains an option for relapse, but is accompanied by significant treatment-related morbidity and mortality (up to 30% of treated patients). Given these poor options, the use of DLI, where isolation of donor T lymphocytes and their subsequent infusion to the recipient in order to enhance immune-mediated anti-tumor activity, has become a common therapy for disease relapse after allogeneic HSCT, although GVT activity of DLI is quite disease-dependent and risks of GVHD remain high. A major goal of transplant biology is to devise strategies to dissociate GVT from GVHD, with focus on both the antigen presenting cells and the effector T cells. For example, immune effector mechanism underlying GVT and GVHD may be different and manipulation of these differences with pharmaceutical agents could lead to a therapeutic benefit to cancer patients.

DLI is effective in treating relapsed chronic myelogenous leukemia (CML) and can induce sustained long-term molecular remission in most patients who relapse with chronic phase disease. However, treatment of accelerated phase or blast crisis CML with DLI is markedly less effective compared to chronic phase CML. Moreover, the activity of DLI is disappointing in acute myelogenous leukemia (AML) when compared with CML. In many cases, it appears the rapid proliferation of AML and high disease burden cannot be managed with GVL induction alone, which can take weeks to fully develop following DLI. Relapsed acute lymphoblastic leukemia has also been notoriously refractory to treatment with DLI in most cases. Prior to the invention described herein, DLI could only result in meaningful GVT effects in a subset of patients with relapsed myeloma. DLI is possibly improved in combination with chemotherapy or immunomodulatory drugs, but toxicity, GVHD, and variable durability of response remain important concerns. Similarly, significant GVT effect has been reported in indolent non-Hodgkin's lymphoma (NHL) following stem cell and DLI therapy, with less evidence supporting a strong effect in more aggressive NHL histologies. DLI has not been extensively studied in chronic lymphocytic leukemia or Hodgkin's lymphoma, but its activity is generally low or of short duration and GVHD is a significant concern. Together, these findings indicate that manipulation of DLI is needed to improve its efficacy and lower its toxicities, particularly for high-risk patients.

Several different factors have been evaluated in DLI treatment of cancer patients. The optimal cell dose and dosing schedule may influence development of GVT and GVHD. There has been concern that outcomes after unrelated donor DLI may be different from those after DLI from an HLA-matched haploidentical donor. With advances in available graft engineering techniques, DLI can now be tailored in attempts to tip the balance of immunity away from GVHD and towards GVT. For example, selective depletion of alloreactive T cells or CD8⁺ T cells or enrichment of CD4⁺ T cells (and Treg cells) or NK cells has been used to reduce GVHD. Strategies to increase the activity of DLI toward the tumors include ex vivo activation prior to transplant. In addition to DLI, adoptive cell therapies using autologous lymphocytes, including tumor-infiltrating lymphocytes, have been studied in a number of clinical settings for both hematologic and solid tumors. T cell engineering has also been developed to introduce suicide genes into the transferred lymphocytes to permit their elimination in the event of GVHD or to introduce chimeric antigen receptors (CAR) or T cell receptors specific to various tumor antigens to direct immune effector activity of the transferred cells against the tumors. Use of immune suppressive and immunomodulatory drugs and monoclonal antibodies post-transplant have also been evaluated. Further strategies that compliment these approaches are required to enhance specificity, maximize GVT, and minimize GVHD after adoptive immunotherapy. As detailed below, the invention described herein addresses these unmet needs.

The adoptive cell therapy approaches described above also typically include pre-transplant conditioning regimen to facilitate engraftment of the transferred cells. Such regimens are known in the art and could include, but are not limited to, myeloablative (MA) conditioning chemo- and radio-therapy, reduced intensity conditioning (RIC) and non-MA regimens. As indicated above, selection of the appropriate conditioning regimen influences TRM, acceptance and persistence of the transplant, GVT activity and GVHD. For the purpose of this disclosure, the conditioning regimen is assumed to be part of the adoptive cell therapy.

Interleukin-15 (IL-15) is a potent cytokine that increases CD8+ T and NK cell numbers and function in experimental models. However, prior to the invention described herein, there were obstacles in using IL-15 therapeutically, specifically its low potency and short in vivo half-life. To help overcome this, an IL-15 superagonist complex (referred to as ALT-803 or IL-15 SA) comprised of an IL-15N72D mutation and IL-15RαSu/Fc fusion was developed. ALT-803 exhibits a significantly longer serum half-life, improved biodistribution to the lymphoid organs, and increased in vivo activity against various tumors in animal models.

As described herein, the effects of ALT-803 in mouse recipients of allogeneic hematopoietic stem cell transplantation were evaluated. As described in detail below, weekly administration of ALT-803 to transplant recipients significantly increased the number of CD8+ T cells (specifically the CD44+ memory/activated phenotype) and NK cells. Levels of CD8+ T cells expressing IFN-γ and TNF-α were increased in ALT-803-treated mice. ALT-803 also upregulated NKG2D expression on CD8+ T cells. Moreover, ALT-803 enhanced proliferation and cytokine secretion of adoptively transferred CFSE-labeled T cells in syngeneic and allogeneic models by specifically stimulating the slow proliferative and non-proliferative cells to become active proliferating cells. As described in detail below, ALT-803's effects on anti-tumor activity against murine mastocytoma (P815) and murine B cell lymphoma (A20) were also evaluated. ALT-803 enhanced graft-versus-tumor (GVT) activity in these tumors following T cell infusion. ALT-803 administration provided GVT activity against A20 lymphoma cells in the murine donor leukocyte infusion (DLI) model without increasing graft-versus-host disease. Thus, as described herein, ALT-803 is a highly potent T-cell lymphoid growth factor and an immunotherapeutic agent to complement stem cell transplantation and adaptive immunotherapy.

IL-15 is a pleiotropic cytokine that plays various roles in the innate and adaptive immune systems, including the development, activation, homing and survival of immune effector cells, especially NK, NK-T and CD8⁺ T cells (Cooper, M. A., et al., Blood, 2001. 97(10): p. 3146-51). IL-15, a member of the common gamma chain (γc) cytokine family, binds to a receptor complex that consists of IL-15Rα, IL-2Rβ and the γc chain (Grabstein, K. H., et al., Science, 1994. 264(5161): p. 965-8; Giri, J. G., et al., Embo J, 1995. 14(15): p. 3654-63). Furthermore, IL-15 functions as a key regulator of development, homeostasis and activity of NK cells (Prlic, M., et al., J Exp Med, 2003. 197(8): p. 967-76; Carson, W. E., et al., J Clin Invest, 1997. 99(5): p. 937-43). IL-15 administration to normal mice or overexpression of IL-15 in the transgenic mouse model increases the number and percentage of NK cells in the spleen (Evans, R., et al., Cell Immunol, 1997. 179(1): p. 66-73; Marks-Konczalik, J., et al., Proc Natl Acad Sci USA, 2000. 97(21): p. 11445-50), the proliferation and survival of NK cells, as well as their cytolytic activity and cytokine secretion. IL-15 administration could also increase the NK cell number and function in recipients of stem cell transplantation (Katsanis, E., et al., Transplantation, 1996. 62(6): p. 872-5; Judge, A. D., et al., J Exp Med, 2002. 196(7): p. 935-46; Alpdogan, O., et al., Blood, 2005. 105(2): p. 865-73; Sauter, C. T., et al., Bone marrow transplantation, 2013. 48(9): p. 1237-42).

The primary limitations in clinical development of recombinant human IL-15 (rhIL-15) are low production yields in standard mammalian cell expression systems and a short serum half-life (Ward, A., et al., Protein Expr Purif, 2009. 68(1): p. 42-8; Bessard, A., et al., Mol Cancer Ther, 2009. 8(9): p. 2736-45). The formation of the IL-15:IL-15Rα complex, with both proteins co-expressed in the same cell can stimulate immune effector cells bearing the IL-2βγc receptor through a trans-presentation mechanism. In addition, when IL-15 is bound to IL-15Rα, it increased the affinity of the IL-15 to IL-2Rβ approximately 150-fold, when compared with free IL-15 (Ring, A. M., et al., Nat Immunol, 2012. 13(12): p. 1187-95). A superagonist mutant of IL-15 (IL-15N72D), which has increased IL-2Rβ binding ability (4-5 fold higher than native IL-15) has been identified for therapeutic usages (Zhu, X., et al., Novel human interleukin-15 agonists. J Immunol, 2009. 183(6): p. 3598-607).

As described in detail below, the strong interaction of IL-15N72D and soluble IL-15Rα was exploited to create an IL-15 superagonist complex with IL-15N72D bound to IL-15RαSu/Fc. The soluble fusion protein, IL-15RαSu/Fc, was created by linking the human IL-15RαSu domain with human IgG1 containing the Fc domain. Studies on IL-15:IL-15Rα complexes show an advantage of increased intracellular stability of IL-15 (Bergamaschi, C., et al., J Biol Chem, 2008. 283(7): p. 4189-99; Duitman, E. H., et al., Mol Cell Biol, 2008. 28(15): p. 4851-61). Co-expression of both the IL-15N72D and IL-15RαSu/Fc proteins resulted in a soluble and stable complex with significantly longer serum half-life and increased biological activity, compared to native IL-15 (Han, K. P., et al., Cytokine, 2011. 56(3): p. 804-10). As indicated above, this IL-15N72D:IL-15RαSu/Fc complex (ALT-803) was >10-fold more active than free IL-15 in promoting in vitro proliferation of IL-15-dependent cells (Zhu, X., et al., Novel human interleukin-15 agonists. J Immunol, 2009. 183(6): p. 3598-607). ALT-803 has potent anti-tumor activity in syngeneic murine models of multiple myeloma (Xu, W., et al., Cancer Res, 2013. 73(10): p. 3075-86). Described herein are the potent effects of ALT-803 on immune reconstitution and graft-versus-tumor (GVT)/graft-versus-leukemia (GVL) activity in recipients of allogeneic hematopoietic stem cell transplantation (HSCT) in murine models.

IL-15 enhances anti-tumor activity in recipients of allogeneic and haploidentical HSCT (Alpdogan, O., et al., Blood, 2005. 105(2): p. 865-73; Sauter, C. T., et al., Bone Marrow Transplant, 2013. 48(9): p. 1237-42). IL-15 half-life is roughly 1 hour after administration and must be administered daily for treatment (Stoklasek, T. A., K. S. Schluns, and L. Lefrancois, J Immunol, 2006. 177(9): p. 6072-80). The long-term effects of recombinant human IL-15 (rhIL-15) have been studied in non-human primates, demonstrating that daily administration of IL-15 for 8-14 days resulted in lymphocytosis and leukocytosis, and white blood cell count returned to normal after discontinuation of IL-15 on day 28 in these studies (Berger, C., et al., Blood, 2009. 114(12): p. 2417-26). Immunological parameters also returned to baseline on day 28 in the same studies. Due to the limitations of the short half-life and daily administration of IL-15, alternative dosing strategies of IL-15 require further assessment in the therapeutic setting.

Conlon et al. reported that recombinant human IL-15 administration resulted in NK and CD8⁺ T cells redistribution, proliferation, activation of NK and CD8⁺ T cells and enhanced inflammatory cytokine production after daily bolus infusion (Conlon, K. C., et al., J Clin Oncol, 2015. 33(1): p. 74-82). The authors also mentioned that alternative dosing strategies have been studied to decrease the toxicity of cytokine. ALT-803 has a better safety profile and longer serum half-life which provides advantages in clinical use.

Described herein are results demonstrating that ALT-803 is a potent immunotherapeutic agent for stimulating NK and CD8⁺ T cells in recipients of allogeneic HSCT. As described in detail below, ALT-803 promoted the expansion of CD8⁺ memory T cells and NK cells, but not CD4⁺ T cells. ALT-803 also significantly increased the levels of NKG2D expression on CD8⁺ T cells. NKG2D, an activating receptor of innate immune cells, is mainly expressed on the surface of NK cells, γδ T cells, and activated CD8⁺ T cells. The NKG2D receptor plays a pivotal role in both innate and adaptive immunity against tumorigenesis and tumor surveillance (Bauer, S., et al., Science, 1999. 285(5428): p. 727-9; Coudert, J. D. and W. Held, Semin Cancer Biol, 2006. 16(5): p. 333-43). It was previously identified that ALT-803 induced memory CD8⁺ T cells to proliferate, upregulate receptors involved in innate immunity, secrete IFN-γ and acquire the ability to kill malignant cells in the absence of antigenic stimulation in murine models of multiple myeloma (Xu, W., et al., Cancer Res, 2013. 73(10): p. 3075-86).

The results described herein demonstrate that ALT-803 has similar effects on the immune cells in the HSCT setting. Thus, it is likely that CD8⁺ T cells with high NKG2D expression (i.e., NKT cells) are induced by ALT-803, and contribute to potent anti-tumor activity in HSCT. The results presented herein are consistent with the results from a recent report that showed NKG2D expression on CD8⁺ T cells is related to mediating GVHD and GVT by promoting the survival and cytotoxic function of CD8⁺ T cells (Karimi, M. A., et al., Blood, 2015). NKG2D blockade was shown to attenuate GVHD, while allowing CD8⁺ T cells to regain anti-tumor activity. Besides functioning as an activating receptor for cell-mediated cytotoxicity of NK and NK-T cells against tumors, NKG2D has also been suggested to act as a receptor to recruit NK and NKT cells to the tumor sites in which tumor cells overexpress stress-inducible NKG2D ligands (Maccalli, C., S. Scaramuzza, and G. Parmiani, Cancer Immunol Immunother, 2009. 58(5): p. 801-8). In the HSCT study described herein, ALT-803 did not significantly promote CD4⁺ T cell proliferation and activation.

IL-15 administration after allogeneic HSCT may enhance the occurrence of GVHD in T cell-depleted models with no effects on GVHD after TCD-BMT (Alpdogan, O., et al., Blood, 2005. 105(2): p. 865-73). Interestingly, IL-15 did not increase GVHD in recipients of a very low dose T cell infusion (Sauter, C. T., et al., Bone Marrow Transplant, 2013. 48(9): p. 1237-42). Using the same model in this study, it was identified that ALT-803 did not increase the occurrence of GVHD and resulted in improved survival of haploidentical HSCT recipients. Described herein are results that demonstrate that ALT-803 increased NK cell numbers in recipients of haploidentical HSCT. ALT-803 also potently activates the cytotoxicity of NK cells (Seay, K., et al., J Virol, 2015. 89(12): p. 6264-74). NK cell alloreactivity in recipients of mismatched HSCT may suppress development of GVHD by decreasing host-derived antigen presenting cells (Ruggeri, L., et al., Science, 2002. 295(5562): p. 2097-100). Thus, it is conceivable that the increase of NK cell numbers and the enhancement of their cytotoxicity by ALT-803 administration in haploidentical HSCT not only contribute to the GVT but also decrease host-derived antigen presenting cells. The decrease in host-derived antigen presenting cells reduces the activation of host-specific CD8⁺ effector T cells which are responsible for GVHD. However, NK cell-associated GVT activity was apparently not strong enough to overcome the A20 tumor cell growth and improve the overall survival of the A20 lymphoma bearing recipient without T cell infusion pre- or post-transplant. Only a low dose of T cell infusion was required to provide survival advantage in the ALT-803 treatment group. This is likely the result of ALT-803's unique capabilities of promoting the expansion of CD8⁺ memory T cells and enhancing their effector functions.

DLI has been developed as a strategy for relapse-management by increasing GVT effects after allogeneic HSCT (Kolb, H. J., et al., Blood, 1990. 76(12): p. 2462-5). DLI is used to treat most malignant hematologic diseases in recipients with relapsed disease after HSCT. The general response rate is less than 30% in patients with acute leukemia and is not durable (Tomblyn, M. and H. M. Lazarus, Bone marrow transplantation, 2008. 42(9): p. 569-79). Collins et al. has found the response rate to DLI to be less than 20% in acute leukemia patients (Collins, R. H., Jr., et al., J Clin Oncol, 1997. 15(2): p. 433-44). In recent years, methods have been developed to enhance the efficacy of DLI for the treatment of relapsed or persistent hematological malignancies after allogeneic HSCT (Chang, Y. J. and X. J. Huang, Blood Rev, 2013. 27(1): p. 55-62). Enhancing donor leukocyte activity with various cytokines has been explored. IL-2 treatment after DLI in patients with relapsed leukemia after allogeneic HSCT did not provide beneficial outcome, and increased the occurrence of GVHD (Inamoto, Y., et al., Biol Blood Marrow Transplant, 2011. 17(9): p. 1308-15). Prior to the invention described herein, IL-15 has not been used following DLI in humans or murine transplant models. Described herein is the development of a DLI model against the A20 murine lymphoma model system. The experiments described herein focused on exploring the activity of purified T cell-containing DLI and results revealed that ALT-803 administration significantly enhanced activity of DLI in murine lymphoma model. Moreover, preliminary results in non-transplant lymphoma models demonstrate that ALT-803 can increase anti-lymphoma activity of autologous T cell infusion in normal mice after lymphodepletion, suggesting that ALT-803 plays an important role in lymphoma/leukemia therapy.

Described herein are results that demonstrate that once a week administration of ALT-803 provides sustained immunological and anti-tumor activities in murine tumor models, murine mastocytoma and murine B cell lymphoma. Substantial anti-tumor activity of ALT-803 has also been previously reported against multiple myeloma in syngeneic models (Xu, W., et al., Cancer Res, 2013. 73(10): p. 3075-86). This is likely due to the longer serum half-life of ALT-803 and its favorable pharmacokinetic profile compared to rhIL-15 (Han, K. P., et al., Cytokine, 2011. 56(3): p. 804-10). The results of these studies support the weekly dosing regimen currently in various clinical trials for solid and hematological malignancies.

In summary, ALT-803 is a potent lymphoid growth factor and is useful as a powerful therapeutic for boosting the immune function in recipients of stem cell transplantation and adaptive T cell therapy without exacerbating GVHD.

### IL-15:IL-15Rα Complex

As defined above, an IL-15:IL-15Rα fusion protein complex can refer to a complex having IL-15 non-covalently bound to the soluble IL-15Rα domain of the native IL-15Rα. In some cases, the soluble IL-15Rα is covalently linked to a biologically active polypeptide and/or to an IgG Fc domain. The IL-15 can be either IL-15 or IL-15 covalently linked to a second biologically active polypeptide. The crystal structure of the IL-15:IL-15Rα complex is shown in Chirifu et al., 2007 Nat Immunol 8, 1001-1007, incorporated herein by reference.

In one aspect, the invention provides a method for making an IL-15:IL-15Rα fusion protein complex, the method involving introducing into a host cell (e.g., a mammalian cell) a first DNA vector encoding IL-15 (or IL-15 variant) and a second DNA vector encoding an IL-15Rα fusion protein; culturing the host cell in media under conditions sufficient to express the IL-15 (or IL-15 variant) and the IL-15Rα fusion protein; and purifying the IL-15:IL-15Rα fusion protein complex from the host cell or media.

In another aspect, the invention provides a method of making an IL-15:IL-15Rα complex containing an IL-15Rα/Fc fusion protein, the method involving introducing into a host cell a first DNA encoding IL-15 (or IL-15 variant) and a second DNA encoding an IL-15Rα/Fc fusion protein; culturing the host cell in media under conditions sufficient to express the IL-15 (or IL-15 variant) and the IL-15Rα/Fc fusion protein; and purifying the IL-15:IL-15Rα/Fc complex from the host cell or media.

In another aspect, the invention provides a method of making an IL-15:IL-15Rα fusion protein complex containing an IL-15Rα/Fc fusion protein, the method involving co-expressing IL-15 (or IL-15 variant) and an IL-15Rα/Fc fusion protein in a host cell; culturing the host cell in media under conditions sufficient to express the IL-15 (or IL-15 variant) and the IL-15Rα/Fc fusion protein; and purifying the IL-15:IL-15Rα/Fc fusion protein complex from the host cell or media.

In another aspect, the invention provides a method of making an IL-15N72D:IL-15RαSu/Fc fusion protein complex involving co-expressing IL-15N72D and an IL-15RαSu/Fc fusion protein in a host cell; culturing the host cell in media under conditions sufficient to express the IL-15N72D and the IL-15RαSu/Fc fusion protein; and purifying the IL-15N72D:IL-15RαSu/Fc fusion protein complex from the host cell or media where both IL-15 binding sites of the IL-15N72D:IL-15RαSu/Fc complex are fully occupied.

In various embodiments of the above aspects or any other aspect of the invention delineated herein, the IL-15Rα fusion protein comprises soluble IL-15Rα, e.g., IL-15Rα covalently linked to a biologically active polypeptide (e.g., the heavy chain constant domain of IgG, an Fc domain of the heavy chain constant domain of IgG). In other embodiments of the invention of the above aspects, IL-15 comprises IL-15, e.g., IL-15 covalently linked to a second biologically active polypeptide. In other embodiments, purifying the IL-15:IL-15Rα complex from the host cell or media involves capturing the IL-15:IL-15Rα complex on an affinity reagent that specifically binds the IL-15:IL-15Rα fusion protein complex. In other embodiments, the IL-15Rα fusion protein contains an IL-15Rα/Fc fusion protein and the affinity reagent specifically binds the Fc domain. In other embodiments, the affinity reagent is Protein A or Protein G. In other embodiments, the affinity reagent is an antibody. In other embodiments, purifying the IL-15:IL-15Rα complex from the host cell or media comprises ion exchange chromatography. In other embodiments, purifying the IL-15:IL-15Rα complex from the host cell or media comprises size exclusion chromatography.

In other embodiments, the IL-15Rα comprises IL-15RαSushi (IL-15RαSu). In other embodiments, the IL-15 is a variant IL-15 (e.g., IL-15N72D). In other embodiments, the IL-15 binding sites of the IL-15:IL-15Rα complex are fully occupied. In other embodiments, both IL-15 binding sites of the IL-15:IL-15RαSu/Fc complex are fully occupied. In other embodiments, the IL-15:IL-15Rα complex is purified based on the complex charge or size properties. In other embodiments, the fully occupied IL-15N72D:IL-15RαSu/Fc fusion protein complex is purified by anion exchange chromatography based on the complex charge properties. In other embodiments, the fully occupied IL-15N72D:IL-15RαSu/Fc fusion protein complex is purified using a quaternary amine-based resin with binding conditions employing low ionic strength neutral pH buffers and elution conditions employing buffers of increasing ionic strength.

In certain embodiments of the soluble fusion protein complexes of the invention, the IL-15 polypeptide is an IL-15 variant having a different amino acid sequence than native IL-15 polypeptide. The human IL-15 polypeptide is referred to herein as huIL-15, hIL-15, huIL15, hIL15, IL-15 wild type (wt) and variants thereof are referred to using the native amino acid, its position in the mature sequence and the variant amino acid. For example, huIL15N72D refers to human IL-15 comprising a substitution of N to D at position 72. In certain embodiments, the IL-15 variant functions as an IL-15 agonist as demonstrated, e.g., by increased binding activity for the IL-15RβγC receptors compared to the native IL-15 polypeptide. In certain embodiments, the IL-15 variant functions as an IL-15 antagonist as demonstrated by e.g., decreased binding activity for the IL-15RβγC receptors compared to the native IL-15 polypeptide. In certain embodiments, the IL-15 variant has increased binding affinity or a decreased binding activity for the IL-15RβγC receptors compared to the native IL-15 polypeptide. In certain embodiments, the sequence of the IL-15 variant has at least one (i.e., 1, 2, 3, 4, 5, 6, 7, 8, 9, 10, or more) amino acid change compared to the native IL-15 sequence. The amino acid change can include one or more of an amino acid substitution or deletion in the domain of IL-15 that interacts with IL-15Rβ and/or IL-15RγC. In certain embodiments, the amino acid change is one or more amino acid substitutions or deletions at position 8, 61, 65, 72, 92, 101, 108, or 111 of the mature human IL-15 sequence. For example, the amino acid change is the substitution of D to N or A at position 8, D to A at position 61, N to A at position 65, N to R at position 72 or Q to A at position 108 of the mature human IL-15 sequence, or any combination of these substitutions. In certain embodiments, the amino acid change is the substitution of N to D at position 72 of the mature human IL-15 sequence.

### ALT-803

ALT-803 comprises an IL-15 mutant with increased ability to bind IL-2Rβγ and enhanced biological activity (U.S. Pat. No. 8,507,222, incorporated herein by reference). This super-agonist mutant of IL-15 was described in a publication (J Immunol 2009 183:3598) and a patent has been issued by the U.S. Patent & Trademark Office on the super agonist and several patents applications are pending (e.g., U.S. Ser. Nos. 12/151,980 and 13/238,925). This IL-15 super-agonist in combination with a soluble IL-15α receptor fusion protein (IL-15RαSu/Fc) results in a protein complex with highly potent IL-15 activity in vitro and in vivo (Han et al., 2011, Cytokine, 56: 804-810; Xu, et al., 2013 Cancer Res. 73:3075-86, Wong, et al., 2013, OncoImmunology 2:e26442). This IL-15 super agonist complex (IL-15N72D:IL-15RαSu/Fc) is referred to as ALT-803. Pharmacokinetic analysis indicated that the complex has a half-life of 25 hours following i.v. administration in mice. ALT-803 exhibits impressive anti-tumor activity against aggressive solid and hematological tumor models in immunocompetent mice. It can be administered as a monotherapy using a twice weekly or weekly i.v. dose regimen or as combinatorial therapy with an antibody. The ALT-803 anti-tumor response is also durable. Tumor-bearing mice that were cured after ALT-803 treatment were also highly resistant to re-challenge with the same tumor cells indicating that ALT-803 induces effective immunological memory responses against the re-introduced tumor cells.

### Interleukin-15

Interleukin-15 (IL-15) is an important cytokine for the development, proliferation, and activation of effector NK cells and CD8⁺ memory T cells. IL-15 binds to the IL-15 receptor α (IL-15Rα) and is presented in trans to the IL-2/IL-15 receptor β-common γ chain (IL-15Rβγ_{c}) complex on effector cells. IL-15 and IL-2 share binding to the IL-15Rβγ_{c}, and signal through STAT3 and STATS pathways. However, IL-2 also supports maintenance of CD4⁺CD25⁺FoxP3⁺ regulatory T (Treg) cells and induces cell death of activated CD8⁺ T cells. These effects may limit the therapeutic activity of IL-2 against tumors. IL-15 does not share these immunosuppressive activities with IL-2. Additionally, IL-15 is the only cytokine known to provide anti-apoptotic signals to effector CD8⁺ T cells. IL-15, either administered alone or as a complex with the IL-15Rα, exhibits potent anti-tumor activities against well-established solid tumors in experimental animal models and, thus, has been identified as one of the most promising immunotherapeutic drugs that could potentially cure cancer.

To facilitate clinical development of an IL-15-based cancer therapeutic, an IL-15 mutant (IL-15N72D) with increased biological activity compared to IL-15 was identified (Zhu et al., J Immunol, 183: 3598-3607, 2009). The pharmacokinetics, biodistribution, and biological activity of this IL-15 super-agonist (IL-15N72D) was further improved by the creation of IL-15N72D:IL-15RαSu/Fc fusion complex (ALT-803), such that the super-agonist complex has at least 25-times the activity of the native cytokine in vivo (Han et al., Cytokine, 56: 804-810, 2011).

### Fc Domain

ALT-803 comprises an IL-15N72D:IL-15RαSu/Fc fusion complex. Fusion proteins that combine the Fc regions of IgG with the domains of another protein, such as various cytokines and soluble receptors have been reported (see, for example, Capon et al., Nature, 337:525-531, 1989; Chamow et al., Trends Biotechnol., 14:52-60, 1996; U.S. Pat. Nos. 5,116,964 and 5,541,087). The prototype fusion protein is a homodimeric protein linked through cysteine residues in the hinge region of IgG Fc, resulting in a molecule similar to an IgG molecule without the heavy chain variable and C_{H1} domains and light chains. The dimeric nature of fusion proteins comprising the Fc domain may be advantageous in providing higher order interactions (i.e. bivalent or bispecific binding) with other molecules. Due to the structural homology, Fc fusion proteins exhibit an in vivo pharmacokinetic profile comparable to that of human IgG with a similar isotype. Immunoglobulins of the IgG class are among the most abundant proteins in human blood, and their circulation half-lives can reach as long as 21 days. To extend the circulating half-life of IL-15 or an IL-15 fusion protein and/or to increase its biological activity, fusion protein complexes containing the IL-15 domain non-covalently bound to IL-15RαSu covalently linked to the Fc portion of the human heavy chain IgG protein have been made (e.g., ALT-803).

The term “Fc” refers to a non-antigen-binding fragment of an antibody. Such an “Fc” can be in monomeric or multimeric form. The original immunoglobulin source of the native Fc is preferably of human origin and may be any of the immunoglobulins, although IgG 1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms. Fc domains containing binding sites for Protein A, Protein G, various Fc receptors and complement proteins.

In some embodiments, the term “Fc variant” refers to a molecule or sequence that is modified from a native Fc, but still comprises a binding site for the salvage receptor, FcRn. International applications WO 97/34631 (published Sep. 25, 1997) and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, in certain embodiments, the term “Fc variant” comprises a molecule or sequence that lacks one or more native Fc sites or residues that affect or are involved in (1) disulfide bond formation, (2) incompatibility with a selected host cell (3)N-terminal heterogeneity upon expression in a selected host cell, (4) glycosylation, (5) interaction with complement, (6) binding to an Fc receptor other than a salvage receptor, (7) antibody-dependent cell-mediated cytotoxicity (ADCC), or (8) antibody dependent cellular phagocytosis (ADCP). Fc variants are described in further detail hereinafter.

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by recombinant gene expression or by other means.

### Fusions Protein Complexes

The invention provides ALT-803, which is a protein complex between IL-15N72D and IL-15RαSu/Fc.

An exemplary IL-15N72D nucleic acid sequence is provided below (with leader peptide) (SEQ ID NO: 1):

(Leader peptide)
atggagacagacacactcctgttatgggtactgctgctctgggttccag
gttccaccggt-
(IL-15N72D)
aactgggtgaatgtaataagtgatttgaaaaaaattgaagatcttattc
aatctatgcatattgatgctactttatatacggaaagtgatgttcaccc
cagttgcaaagtaacagcaatgaagtgctttctcttggagttacaagtt
atttcacttgagtccggagatgcaagtattcatgatacagtagaaaatc
tgatcatcctagcaaacgacagtttgtcttctaatgggaatgtaacaga
atctggatgcaaagaatgtgaggaactggaggaaaaaaatattaaagaa
tttttgcagagttttgtacatattgtccaaatgttcatcaacacttct
(Stop codon)
taa

An exemplary IL-15N72D amino acid sequence is provided below (with leader peptide) (SEQ ID NO: 2):

(Leader peptide)
METDTLLLWVLLLWVPGSTG-
(IL-15N72D)
NWVNVISDLKKIEDLIQSMHIDATLYTESDVHPSCKVTAMKCELLELQVI
SLESGDASIHDTVENLIILANDSLSSNGNVTESGCKECEELEEKNIKEFL
QSFVHIVQMFINTS
In some cases, the leader peptide is cleaved from
the mature IL-15N72D polypeptide
(SEQ ID NO: 3):
(IL-15N72D)
NWVNVISDLKKIEDLIQSMHIDATLYTESDVHPSCKVTAMKCFLLELQVI
SLESGDASIHDTVENLIILANDSLSSNGNVTESGCKECEELEEKNIKEFL
QSFVHIVQMFINTS

An exemplary IL-15RαSu/Fc nucleic acid sequence (with leader peptide) is provided below (SEQ ID NO: 4):

(Leader peptide)
atggacagacttacttettcattectgctcctgattgtccctgcgtacgtc
ttgtcc-
(IL-15RaSu)
atcacgtgccctccccccatgtccgtggaacacgcagacatctgggtcaag
agctacagcttgtactccagggagcggtacatttgtaactctggtttcaag
cgtaaagccggcacgtccagcctgacggagtgcgtgttgaacaaggccacg
aatgtcgcccactggacaacccccagtctcaaatgtattaga-
(IgG1 CH2-CH3 (Fc domain))
gagcccaaatcttgtgacaaaactcacacatgcccaccgtgcccagcacct
gaactcctggggggaccgtcagtcttcctcttccccccaaaacccaaggac
accctcatgatctcccggacccctgaggtcacatgcgtggtggtggacgtg
agccacgaagaccctgaggtcaagttcaactggtacgtggacggcgtggag
gtgcataatgccaagacaaagccgcgggaggagcagtacaacagcacgtac
cgtgtggtcagcgtcctcaccgtcctgcaccaggactggctgaatggcaag
gagtacaagtgcaaggtctccaacaaagccctcccagcccccatcgagaaa
accatctccaaagccaaagggcagccccgagaaccacaggtgtacaccctg
cccccatcccgggatgagctgaccaagaaccaggtcagcctgacctgcctg
gtcaaaggcttctatcccagcgacatcgccgtggagtgggagagcaatggg
cagccggagaacaactacaagaccacgcctcccgtgctggactccgacggc
tccttcttcctctacagcaagctcaccgtggacaagagcaggtggcagcag
gggaacgtcttctcatgctccgtgatgcatgaggctctgcacaaccactac
acgcagaagagcctctccctgtctccgggtaaa-
(Stop codon)
taa

An exemplary IL-15RαSu/Fc amino acid sequence (with leader peptide) is provided below (SEQ ID NO: 5):

(Leader peptide)
MDRLTSSFLLLIVPAYVLS-
(IL-15RaSu)
ITCPPPMSVEHADIWVKSYSLYSRERYICNSGFKRKAGTSSLTECVLNKAT
NVAHWTTPSLKCIR-
(IgG1 CH2-CH3 (Fc domain))
EPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQ
GNVFSCSVMHEALHNHYTQKSLSLSPGK

In some cases, the mature IL-15RαSu/Fc protein lacks the leader sequence (SEQ ID NO: 6):

(IL-15RaSu)
ITCPPPMSVEHADIWVKSYSLYSRERYICNSGFKRKAGTSSLTECVLNKA
TNVAHWTTPSLKCIR-
(IgG1 CH2-CH3 (Fc domain))
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

In certain embodiments, the ALT-803 polypeptides could serve as a scaffold for fusion to other protein domains. In such fusion protein complexes, a first fusion protein comprises a first biologically active polypeptide covalently linked to interleukin-15 (IL-15) or functional fragment thereof; and the second fusion protein comprises a second biologically active polypeptide covalently linked to soluble interleukin-15 receptor alpha (IL-15Rα) polypeptide or functional fragment thereof, where the IL-15 domain of a first fusion protein binds to the soluble IL-15Rα domain of the second fusion protein to form a soluble fusion protein complex. Fusion protein complexes of the invention also comprise immunoglobulin Fc domain or a functional fragment thereof linked to one or both of the first and second fusion proteins. Preferably, the Fc domains linked to the first and second fusion proteins interact to form a fusion protein complex. Such a complex may be stabilized by disulfide bond formation between the immunoglobulin Fc domains. In certain embodiments, the soluble fusion protein complexes of the invention include an IL-15 polypeptide, IL-15 variant or a functional fragment thereof and a soluble IL-15Rα polypeptide or a functional fragment thereof, wherein one or both of the IL-15 and IL-15Rα polypeptides further include an immunoglobulin Fc domain or a functional fragment thereof.

In a further embodiment, one or both of the first and second biologically active polypeptides comprises an antibody or functional fragment thereof.

In another embodiment, the antigen for the antibody domain comprises a cell surface receptor or ligand.

In a further embodiment, the antigen comprises a CD antigen, cytokine or chemokine receptor or ligand, growth factor receptor or ligand, tissue factor, cell adhesion molecule, WIC/WIC-like molecules, Fc receptor, Toll-like receptor, NK receptor, TCR, BCR, positive/negative co-stimulatory receptor or ligand, death receptor or ligand, tumor associated antigen, or virus encoded antigen.

As used herein, the term “biologically active polypeptide” or “effector molecule” is meant an amino acid sequence such as a protein, polypeptide or peptide; a sugar or polysaccharide; a lipid or a glycolipid, glycoprotein, or lipoprotein that can produce the desired effects as discussed herein. Effector molecules also include chemical agents. Also contemplated are effector molecule nucleic acids encoding a biologically active or effector protein, polypeptide, or peptide. Thus, suitable molecules include regulatory factors, enzymes, antibodies, or drugs as well as DNA, RNA, and oligonucleotides. The biologically active polypeptides or effector molecule can be naturally-occurring or it can be synthesized from known components, e.g., by recombinant or chemical synthesis and can include heterologous components. A biologically active polypeptides or effector molecule is generally between about 0.1 to 100 KD or greater up to about 1000 KD, preferably between about 0.1, 0.2, 0.5, 1, 2, 5, 10, 20, 30 and 50 KD as judged by standard molecule sizing techniques such as centrifugation or SDS-polyacrylamide gel electrophoresis. Desired effects of the invention include, but are not limited to, for example, forming a fusion protein complex of the invention with increased binding activity, killing a target cell, e.g. either to induce cell proliferation or cell death, initiate an immune response, in preventing or treating a disease, or to act as a detection molecule for diagnostic purposes. For such detection, an assay could be used, for example an assay that includes sequential steps of culturing cells to proliferate same.

Covalently linking the effector molecule to the fusion protein complexes of the invention in accordance with the invention provides a number of significant advantages. Fusion protein complexes of the invention can be produced that contain a single effector molecule, including such a peptide of known structure. Additionally, a wide variety of effector molecules can be produced in similar DNA vectors. That is, a library of different effector molecules can be linked to the fusion protein complexes for recognition of infected or diseased cells. Further, for therapeutic applications, rather than administration of a the fusion protein complex of the invention to a subject, a DNA expression vector coding for the fusion protein complex can be administered for in vivo expression of the fusion protein complex. Such an approach avoids costly purification steps typically associated with preparation of recombinant proteins and avoids the complexities of antigen uptake and processing associated with conventional approaches.

As noted, components of the fusion proteins and antibodies disclosed herein, e.g., effector molecule conjugates such as cytokines, chemokines, growth factors, protein toxins, immunoglobulin domains or other bioactive molecules and any peptide linkers, can be organized in nearly any fashion provided that the fusion protein or antibody has the function for which it was intended. In particular, each component of the fusion protein can be spaced from another component by at least one suitable peptide linker sequence if desired. Additionally, the fusion proteins may include tags, e.g., to facilitate modification, identification and/or purification of the fusion protein.

### Pharmaceutical Therapeutics

The invention provides pharmaceutical compositions comprising ALT-803 for use as a therapeutic. In one aspect, ALT-803 is administered systemically, for example, formulated in a pharmaceutically-acceptable buffer such as physiological saline. Preferable routes of administration include, for example, instillation into the bladder, subcutaneous, intravenous, intraperitoneal, intramuscular, or intradermal injections that provide continuous, sustained levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia or infection. Generally, amounts will be in the range of those used for other agents used in the treatment of other diseases associated with neoplasia or infection, although in certain instances lower amounts will be needed because of the increased specificity of the compound. A compound is administered at a dosage that enhances an immune response of a subject, or that reduces the proliferation, survival, or invasiveness of a neoplastic cell as determined by a method known to one skilled in the art. Alternatively, the compound is administered at a dosage that reduces infection by a virus or other pathogen of interest.

### Formulation of Pharmaceutical Compositions

The administration of ALT-803 for the treatment of a neoplasia or an infection may be by any suitable means that results in a concentration of the therapeutic that, combined with other components, is effective in ameliorating, reducing, or stabilizing a neoplasia or infection. ALT-803 may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intravesicularly or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice or nonhuman primates, as a skilled artisan recognizes it is routine in the art to modify the dosage for humans compared to animal models. In certain embodiments it is envisioned that the dosage may vary from between about 0.1 μg compound/kg body weight to about 5000 μg compound/kg body weight; or from about 1 μg/kg body weight to about 4000 μg/kg body weight or from about 10 μg/kg body weight to about 3000 μg/kg body weight. In other embodiments this dose may be about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 μg/kg body weight. In other embodiments, it is envisaged that doses may be in the range of about 0.5 μg compound/kg body weight to about 20 μg compound/kg body weight. In other embodiments the doses may be about 0.5, 1, 3, 6, 10, or 20 mg/kg body weight. Of course, this dosage amount may be adjusted upward or downward, as is routinely done in such treatment protocols, depending on the results of the initial clinical trials and the needs of a particular patient.

In particular embodiments, ALT-803 are formulated in an excipient suitable for parenteral administration. In particular embodiments, ALT-803 is administered at 0.5 μg/kg-about 15 μg/kg (e.g., 0.5, 1, 3, 5, 10, or 15 μg/kg).

For the treatment of bladder cancer, ALT-803 is administered by instillation into the bladder. Methods of instillation are known. See, for example, Lawrencia, et al., Gene Ther 8, 760-8 (2001); Nogawa, et al., J Clin Invest 115, 978-85 (2005); Ng, et al., Methods Enzymol 391, 304-13 2005; Tyagi, et al., J Urol 171, 483-9 (2004); Trevisani, et al., J Pharmacol Exp Ther 309, 1167-73 (2004); Trevisani, et al., Nat Neurosci 5, 546-51 (2002)); (Segal, et al., 1975). (Dyson, et al., 2005). (Batista, et al., 2005; Dyson, et al., 2005). In certain embodiments, it is envisioned that the ALT-803 dosage for instillation may vary between about 5 and 1000 μg/dose. In other embodiments the intravesical doses may be about 25, 50, 100, 200, or 400 μg/dose. In other embodiments, ALT-803 is administered by instillation into the bladder in combination with standard therapies, including mitomycin C or Bacille Calmette-Guerin (BCG).

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition comprising ALT-803 may be administered parenterally by injection, infusion or implantation (subcutaneous, intravenous, intramuscular, intravesicularly, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions comprising ALT-803 for parenteral use may be provided in unit dosage forms (e.g., in single-dose ampoules, syringes or bags), or in vials containing several doses and in which a suitable preservative may be added (see below). The composition may be in the form of a solution, a suspension, an emulsion, an infusion device, or a delivery device for implantation or it may be presented as a dry powder to be reconstituted with water or another suitable vehicle before use. Apart from the active agent that reduces or ameliorates a neoplasia or infection, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions comprising ALT-803 may be in a form suitable for sterile injection. To prepare such a composition, the suitable active antineoplastic/anti-infective therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a neoplastic or infectious disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, etc.). ALT-803 may be used in the treatment of any other disorders in which an increase in an immune response is desired.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with neoplasia or infection in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Combination Therapies

In some cases, ALT-803 is administered in combination with an anti-neoplasia or anti-infectious therapeutic such as an antibody, e.g., a tumor-specific antibody. The antibody and ALT-803 may be administered simultaneously or sequentially. In some embodiments, the antibody treatment is an established therapy for the disease indication and addition of ALT-803 treatment to the antibody regimen improves the therapeutic benefit to the patients. Such improvement could be measured as increased responses on a per patient basis or increased responses in the patient population. Combination therapy could also provide improved responses at lower or less frequent doses of antibody resulting in a better tolerated treatment regimen. As indicated, the combined therapy of ALT-803 and an antibody could provide enhances clinical activity through various mechanisms, including augmented ADCC, ADCP, and/or NK cell, T-cell, neutrophil or monocytic cell levels or immune responses.

If desired, ALT-803 is administered in combination with any conventional therapy, including but not limited to, surgery, radiation therapy, chemotherapy, protein-based therapy or biological therapy. Chemotherapeutic drugs include alkylating agents (e.g., platinum-based drugs, tetrazines, aziridines, nitrosoureas, nitrogen mustards), anti-metabolites (e.g., anti-folates, fluoropyrimidines, deoxynucleoside analogues, thiopurines), anti-microtubule agents (e.g., vinca alkaloids, taxanes), topoisomerase inhibitors (e.g., topoisomerase I and II inhibitors), cytotoxic antibiotics (e.g., anthracyclines), protein kinase inhibitors (e.g., tyrosine kinase inhibitors), and immunomodulatory drugs (e.g., thalidomide and analogs).

In other embodiments, ALT-803 is administered in conjunction with adoptive cell therapies or transplants. Such therapies include, but not limited to, allogeneic and autologous hematopoietic stem cell transplantation, donor lymphocyte infusion (DLI), adoptive transfer of tumor infiltrating lymphocytes, or engineered T cells or NK cells including those containing suicide genes, genes from chimeric antigen receptors or TCR specific to tumor antigens, or other genes to facilitate cell proliferation, survival, persistence or activity against the tumor. The transferred cells could be obtained from various sources including the recipient (autologous) or related or unrelated donors. Combination therapy with ALT-803 could be done in vivo, ex vivo or in vitro, or combinations thereof.

### Kits or Pharmaceutical Systems

Pharmaceutical compositions comprising ALT-803 may be assembled into kits or pharmaceutical systems for use in treating a neoplasia. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using ALT-803.

### Recombinant Protein Expression

In general, preparation of the fusion protein complexes of the invention (e.g., components of ALT-803) can be accomplished by procedures disclosed herein and by recognized recombinant DNA techniques.

In general, recombinant polypeptides are produced by transformation of a suitable host cell with all or part of a polypeptide-encoding nucleic acid molecule or fragment thereof in a suitable expression vehicle. Those skilled in the field of molecular biology will understand that any of a wide variety of expression systems may be used to provide the recombinant protein. The precise host cell used is not critical to the invention. A recombinant polypeptide may be produced in virtually any eukaryotic host (e.g., Saccharomyces cerevisiae, insect cells, e.g., Sf21 cells, or mammalian cells, e.g., NIH 3T3, HeLa, COS or preferably CHO cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transfection and the choice of expression vehicle will depend on the host system selected. Transformation methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exists for the production of recombinant polypeptides. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

Once the recombinant polypeptide is expressed, it is isolated, e.g., using affinity chromatography. In one example, an antibody (e.g., produced as described herein) raised against the polypeptide may be attached to a column and used to isolate the recombinant polypeptide. Lysis and fractionation of polypeptide-harboring cells prior to affinity chromatography may be performed by standard methods (see, e.g., Ausubel et al., supra). Once isolated, the recombinant protein can, if desired, be further purified by high performance liquid chromatography (see, e.g., Fisher, Laboratory Techniques in Biochemistry and Molecular Biology, eds., Work and Burdon, Elsevier, 1980).

As used herein, biologically active polypeptides or effector molecules of the invention may include factors such as cytokines, chemokines, growth factors, protein toxins, immunoglobulin domains or other bioactive proteins such as enzymes. Also biologically active polypeptides may include conjugates to other compounds such as non-protein toxins, cytotoxic agents, chemotherapeutic agents, detectable labels, radioactive materials and such.

Cytokines of the invention are defined by any factor produced by cells that affect other cells and are responsible for any of a number of multiple effects of cellular immunity. Examples of cytokines include but are not limited to the IL-2 family, interferon (IFN), IL-10, IL-1, IL-17, TGF and TNF cytokine families, and to IL-1 through IL-35, IFN-α, IFN-β, IFNγ, TGF-β, TNF-α, and TNFβ.

In an aspect of the invention, the first fusion protein comprises a first biologically active polypeptide covalently linked to interleukin-15 (IL-15) domain or a functional fragment thereof. IL-15 is a cytokine that affects T-cell activation and proliferation. IL-15 activity in affecting immune cell activation and proliferation is similar in some respects to IL-2, although fundamental differences have been well characterized (Waldmann, T A, 2006, Nature Rev. Immunol. 6:595-601).

In another aspect of the invention, the first fusion protein comprises an interleukin-15 (IL-15) domain that is an IL-15 variant (also referred to herein as IL-15 mutant). The IL-15 variant preferably comprises a different amino acid sequence that the native (or wild type) IL-15 protein. The IL-15 variant preferably binds the IL-15Rα polypeptide and functions as an IL-15 agonist or antagonist. Preferably, IL-15 variants with agonist activity have super agonist activity. In some embodiments, the IL-15 variant can function as an IL-15 agonist or antagonist independent of its association with IL-15Rα. IL-15 agonists are exemplified by comparable or increased biological activity compared to wild type IL-15. IL-15 antagonists are exemplified by decreased biological activity compared to wild type IL-15 or by the ability to inhibit IL-15-mediated responses. In some examples, the IL-15 variant binds with increased or decreased activity to the IL-2/15RβγC receptors. In some embodiments, the sequence of the IL-15 variant has at least one amino acid change, e.g. substitution or deletion, compared to the native IL-15 sequence, such changes resulting in IL-15 agonist or antagonist activity. Preferably the amino acid substitutions/deletions are in the domains of IL-15 that interact with IL-15Rβ and/or γC. More preferably, the amino acid substitutions/deletions do not affect binding to the IL-15Rα polypeptide or the ability to produce the IL-15 variant. Suitable amino acid substitutions/deletions to generate IL-15 variants can be identified based on putative or known IL-15 structures, comparisons of IL-15 with homologous molecules such as IL-2 with known structure, through rational or random mutagenesis and functional assays, as provided herein, or other empirical methods. Additionally suitable amino acid substitutions can be conservative or non-conservative changes and insertions of additional amino acids. Preferably, IL-15 variants of the invention contain one or more than one amino acid substitutions/deletions at position 6, 8, 10, 61, 65, 72, 92, 101, 104, 105, 108, 109, 111, or 112 of the mature human IL-15 sequence; particularly, D8N (“D8” refers to the amino acid and residue position in the native mature human IL-15 sequence and “N” refers to the substituted amino acid residue at that position in the IL-15 variant), I6S, D8A, D61A, N65A, N72R, V104P or Q108A substitutions result in IL-15 variants with antagonist activity and N72D substitutions result in IL-15 variants with agonist activity.

Chemokines, similar to cytokines, are defined as any chemical factor or molecule which when exposed to other cells are responsible for any of a number of multiple effects of cellular immunity. Suitable chemokines may include but are not limited to the CXC, CC, C, and CX.sub.3C chemokine families and to CCL-1 through CCL-28, CXC-1 through CXC-17, XCL-1, XCL-2, CX3CL1, MIP-1b, IL-8, MCP-1, and Rantes.

Growth factors include any molecules which when exposed to a particular cell induce proliferation and/or differentiation of the affected cell. Growth factors include proteins and chemical molecules, some of which include: GM-CSF, G-CSF, human growth factor and stem cell growth factor. Additional growth factors may also be suitable for uses described herein.

Toxins or cytotoxic agents include any substance that has a lethal effect or an inhibitory effect on growth when exposed to cells. More specifically, the effector molecule can be a cell toxin of, e.g., plant or bacterial origin such as, e.g., diphtheria toxin (DT), shiga toxin, abrin, cholera toxin, ricin, saporin, pseudomonas exotoxin (PE), pokeweed antiviral protein, or gelonin. Biologically active fragments of such toxins are well known in the art and include, e.g., DT A chain and ricin A chain. Additionally, the toxin can be an agent active at the cell surface such as, e.g., phospholipase enzymes (e.g., phospholipase C).

Further, the effector molecule can be a chemotherapeutic drug such as, e.g., vindesine, vincristine, vinblastin, methotrexate, adriamycin, bleomycin, or cisplatin.

Additionally, the effector molecule can be a detectably-labeled molecule suitable for diagnostic or imaging studies. Such labels include biotin or streptavidin/avidin, a detectable nanoparticles or crystal, an enzyme or catalytically active fragment thereof, a fluorescent label such as green fluorescent protein, FITC, phycoerythrin, cychome, texas red or quantum dots; a radionuclide e.g., iodine-131, yttrium-90, rhenium-188 or bismuth-212; a phosphorescent or chemiluminescent molecules or a label detectable by PET, ultrasound or MRI such as Gd- or paramagnetic metal ion-based contrast agents. See e.g., Moskaug, et al. J. Biol. Chem. 264, 15709 (1989); Pastan, I. et al. Cell 47, 641, 1986; Pastan et al., Recombinant Toxins as Novel Therapeutic Agents, Ann. Rev. Biochem. 61, 331, (1992); “Chimeric Toxins” Olsnes and Phil, Pharmac. Ther., 25, 355 (1982); published PCT application no. WO 94/29350; published PCT application no. WO 94/04689; published PCT application no. WO2005046449 and U.S. Pat. No. 5,620,939 for disclosure relating to making and using proteins comprising effectors or tags.

A protein fusion or conjugate complex that includes a covalently linked IL-15 and IL-15Rα domains has several important uses. Cells or tissue susceptible to being damaged or killed can be readily assayed by the methods disclosed herein.

The IL-15 and IL-15Rα polypeptides of the invention suitably correspond in amino acid sequence to naturally occurring IL-15 and IL-15Rα molecules, e.g. IL-15 and IL-15Rα molecules of a human, mouse or other rodent, or other mammal. Sequences of these polypeptides and encoding nucleic acids are known in the literature, including human interleukin 15 (IL15) mRNA—GenBank: U14407.1 (incorporated herein by reference), Mus musculus interleukin 15 (IL15) mRNA—GenBank: U14332.1 (incorporated herein by reference), human interleukin-15 receptor alpha chain precursor (IL15RA) mRNA—GenBank: U31628.1 (incorporated herein by reference), Mus musculus interleukin 15 receptor, alpha chain—GenBank: BC095982.1 (incorporated herein by reference).

In some settings, it can be useful to make the protein fusion or conjugate complexes of the present invention polyvalent, e.g., to increase the valency of the sc-TCR or sc-antibody. In particular, interactions between the IL-15 and IL-15Rα domains of the fusion protein complex provide a means of generating polyvalent complexes. In addition, the polyvalent fusion protein can made by covalently or non-covalently linking together between one and four proteins (the same or different) by using e.g., standard biotin-streptavidin labeling techniques, or by conjugation to suitable solid supports such as latex beads. Chemically cross-linked proteins (for example cross-linked to nanoparticles) are also suitable polyvalent species. For example, the protein can be modified by including sequences encoding tag sequences that can be modified such as the biotinylation BirA tag or amino acid residues with chemically reactive side chains such as Cys or His. Such amino acid tags or chemically reactive amino acids may be positioned in a variety of positions in the fusion protein or antibody, preferably distal to the active site of the biologically active polypeptide or effector molecule. For example, the C-terminus of a soluble fusion protein can be covalently linked to a tag or other fused protein which includes such a reactive amino acid(s). Suitable side chains can be included to chemically link two or more fusion proteins to a suitable nanoparticle to give a multivalent molecule. Exemplary nanoparticles include dendrimers, liposomes, core-shell particles or protein- or PLGA-based particles.

In another embodiment of the invention, one or both of the polypeptides of the fusion protein complex comprises an immunoglobulin domain. Alternatively, the protein binding domain-IL-15 fusion protein can be further linked to an immunoglobulin domain. The preferred immunoglobulin domains comprise regions that allow interaction with other immunoglobulin domains to form multichain proteins as provided above. For example, the immunoglobulin heavy chain regions, such as the IgG1 C_{H2}-C_{H3}, are capable of stably interacting to create the Fc region. Preferred immunoglobulin domains including Fc domains also comprise regions with effector functions, including Fc receptor or complement protein binding activity, and/or with glycosylation sites. In some embodiments, the immunoglobulin domains of the fusion protein complex contain mutations that reduce or augment Fc receptor or complement binding activity or glycosylation, thereby affecting the biological activity of the resulting protein. For example, immunoglobulin domains containing mutations that reduce binding to Fc receptors could be used to generate fusion protein complex of the invention with lower binding activity to Fc receptor-bearing cells, which may be advantageous for reagents designed to recognize or detect specific antigens.

### Nucleic Acids and Vectors

The invention further provides nucleic acid sequences and particularly DNA sequences that encode the present proteins (e.g., components of ALT-803). Preferably, the DNA sequence is carried by a vector suited for extrachromosomal replication such as a phage, virus, plasmid, phagemid, cosmid, YAC, or episome. In particular, a DNA vector that encodes a desired fusion protein can be used to facilitate preparative methods described herein and to obtain significant quantities of the fusion protein. The DNA sequence can be inserted into an appropriate expression vector, i.e., a vector that contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used. See, Sambrook et al., supra and Ausubel et al. supra.

Included in the invention are methods for making a soluble fusion protein complex, the method comprising introducing into a host cell a DNA vector as described herein encoding the first and second fusion proteins, culturing the host cell in media under conditions sufficient to express the fusion proteins in the cell or the media and allow association between IL-15 domain of a first fusion protein and the soluble IL-15Rα domain of a second fusion protein to form the soluble fusion protein complex, purifying the soluble fusion protein complex from the host cells or media.

In general, a preferred DNA vector according to the invention comprises a nucleotide sequence linked by phosphodiester bonds comprising, in a 5′ to 3′ direction a first cloning site for introduction of a first nucleotide sequence encoding a biologically active polypeptide, operatively linked to a sequence encoding an effector molecule.

The fusion protein components encoded by the DNA vector can be provided in a cassette format. By the term “cassette” is meant that each component can be readily substituted for another component by standard recombinant methods. In particular, a DNA vector configured in a cassette format is particularly desirable when the encoded fusion complex is to be used against pathogens that may have or have capacity to develop serotypes.

To make the vector coding for a fusion protein complex, the sequence coding for the biologically active polypeptide is linked to a sequence coding for the effector peptide by use of suitable ligases. DNA coding for the presenting peptide can be obtained by isolating DNA from natural sources such as from a suitable cell line or by known synthetic methods, e.g. the phosphate triester method. See, e.g., Oligonucleotide Synthesis, IRL Press (M. J. Gait, ed., 1984). Synthetic oligonucleotides also may be prepared using commercially available automated oligonucleotide synthesizers. Once isolated, the gene coding for the biologically active polypeptide can be amplified by the polymerase chain reaction (PCR) or other means known in the art. Suitable PCR primers to amplify the biologically active polypeptide gene may add restriction sites to the PCR product. The PCR product preferably includes splice sites for the effector peptide and leader sequences necessary for proper expression and secretion of the biologically active polypeptide-effector fusion complex. The PCR product also preferably includes a sequence coding for the linker sequence, or a restriction enzyme site for ligation of such a sequence.

The fusion proteins described herein are preferably produced by standard recombinant DNA techniques. For example, once a DNA molecule encoding the biologically active polypeptide is isolated, sequence can be ligated to another DNA molecule encoding the effector polypeptide. The nucleotide sequence coding for a biologically active polypeptide may be directly joined to a DNA sequence coding for the effector peptide or, more typically, a DNA sequence coding for the linker sequence as discussed herein may be interposed between the sequence coding for the biologically active polypeptide and the sequence coding for the effector peptide and joined using suitable ligases. The resultant hybrid DNA molecule can be expressed in a suitable host cell to produce the fusion protein complex. The DNA molecules are ligated to each other in a 5′ to 3′ orientation such that, after ligation, the translational frame of the encoded polypeptides is not altered (i.e., the DNA molecules are ligated to each other in-frame). The resulting DNA molecules encode an in-frame fusion protein.

Other nucleotide sequences also can be included in the gene construct. For example, a promoter sequence, which controls expression of the sequence coding for the biologically active polypeptide fused to the effector peptide, or a leader sequence, which directs the fusion protein to the cell surface or the culture medium, can be included in the construct or present in the expression vector into which the construct is inserted. An immunoglobulin or CMV promoter is particularly preferred.

In obtaining variant biologically active polypeptide, IL-15, IL-15Rα or Fc domain coding sequences, those of ordinary skill in the art will recognize that the polypeptides may be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein. In other instance, modifications to amino acid positions can be made to reduce or enhance the biological activity of the protein. Such changes can be introduced randomly or via site-specific mutations based on known or presumed structural or functional properties of targeted residue(s). Following expression of the variant protein, the changes in the biological activity due to the modification can be readily assessed using binding or functional assays.

Homology between nucleotide sequences can be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. For instance, for sequences with about 55% G-C content, hybridization and wash conditions of 40-50° C., 6×SSC (sodium chloride/sodium citrate buffer) and 0.1% SDS (sodium dodecyl sulfate) indicate about 60-70% homology, hybridization and wash conditions of 50-65° C., 1×SSC and 0.1% SDS indicate about 82-97% homology, and hybridization and wash conditions of 52° C., 0.1×SSC and 0.1% SDS indicate about 99-100% homology. A wide range of computer programs for comparing nucleotide and amino acid sequences (and measuring the degree of homology) are also available, and a list providing sources of both commercially available and free software is found in Ausubel et al. (1999). Readily available sequence comparison and multiple sequence alignment algorithms are, respectively, the Basic Local Alignment Search Tool (BLAST) (Altschul et al., 1997) and ClustalW programs. BLAST is available on the World Wide Web at ncbi.nlm.nih.gov and a version of ClustalW is available at 2.ebi.ac.uk.

The components of the fusion protein can be organized in nearly any order provided each is capable of performing its intended function. For example, in one embodiment, the biologically active polypeptide is situated at the C or N terminal end of the effector molecule.

Preferred effector molecules of the invention will have sizes conducive to the function for which those domains are intended. The effector molecules of the invention can be made and fused to the biologically active polypeptide by a variety of methods including well-known chemical cross-linking methods. See, e.g., Means, G. E. and Feeney, R. E. (1974) in Chemical Modification of Proteins, Holden-Day. See also, S. S. Wong (1991) in Chemistry of Protein Conjugation and Cross-Linking, CRC Press. However it is generally preferred to use recombinant manipulations to make the in-frame fusion protein.

As noted, a fusion molecule or a conjugate molecule in accord with the invention can be organized in several ways. In an exemplary configuration, the C-terminus of the biologically active polypeptide is operatively linked to the N-terminus of the effector molecule. That linkage can be achieved by recombinant methods if desired. However, in another configuration, the N-terminus of the biologically active polypeptide is linked to the C-terminus of the effector molecule.

Alternatively, or in addition, one or more additional effector molecules can be inserted into the biologically active polypeptide or conjugate complexes as needed.

### Vectors and Expression

A number of strategies can be employed to express ALT-803. For example, a construct encoding ALT-803 can be incorporated into a suitable vector using restriction enzymes to make cuts in the vector for insertion of the construct followed by ligation. The vector containing the gene construct is then introduced into a suitable host for expression of the fusion protein. See, generally, Sambrook et al., supra. Selection of suitable vectors can be made empirically based on factors relating to the cloning protocol. For example, the vector should be compatible with, and have the proper replicon for the host that is being employed. The vector must be able to accommodate the DNA sequence coding for the fusion protein complex that is to be expressed. Suitable host cells include eukaryotic and prokaryotic cells, preferably those cells that can be easily transformed and exhibit rapid growth in culture medium. Specifically preferred hosts cells include prokaryotes such as E. coli, Bacillus subtillus, etc. and eukaryotes such as animal cells and yeast strains, e.g., S. cerevisiae. Mammalian cells are generally preferred, particularly J558, NSO, SP2-O or CHO. Other suitable hosts include, e.g., insect cells such as Sf9. Conventional culturing conditions are employed. See, Sambrook, supra. Stable transformed or transfected cell lines can then be selected. Cells expressing a fusion protein complex of the invention can be determined by known procedures. For example, expression of a fusion protein complex linked to an immunoglobulin can be determined by an ELISA specific for the linked immunoglobulin and/or by immunoblotting. Other methods for detecting expression of fusion proteins comprising biologically active polypeptides linked to IL-15 or IL-15Rα domains are disclosed in the Examples.

As mentioned generally above, a host cell can be used for preparative purposes to propagate nucleic acid encoding a desired fusion protein. Thus, a host cell can include a prokaryotic or eukaryotic cell in which production of the fusion protein is specifically intended. Thus host cells specifically include yeast, fly, worm, plant, frog, mammalian cells and organs that are capable of propagating nucleic acid encoding the fusion. Non-limiting examples of mammalian cell lines which can be used include CHO dhfr-cells (Urlaub and Chasm, Proc. Natl. Acad. Sci. USA, 77:4216 (1980)), 293 cells (Graham et al., J Gen. Virol., 36:59 (1977)) or myeloma cells like SP2 or NSO (Galfre and Milstein, Meth. Enzymol., 73(B):3 (1981)).

Host cells capable of propagating nucleic acid encoding a desired fusion protein comples encompass non-mammalian eukaryotic cells as well, including insect (e.g., Sp. frugiperda), yeast (e.g., S. cerevisiae, S. pombe, P. pastoris., K. lactis, H. polymorphs, as generally reviewed by Fleer, R., Current Opinion in Biotechnology, 3(5):486496 (1992)), fungal and plant cells. Also contemplated are certain prokaryotes such as E. coli and Bacillus.

Nucleic acid encoding a desired fusion protein can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration. Suitable methods for transfecting host cells can be found in Sambrook et al. supra, and other laboratory textbooks.

Various promoters (transcriptional initiation regulatory region) may be used according to the invention. The selection of the appropriate promoter is dependent upon the proposed expression host. Promoters from heterologous sources may be used as long as they are functional in the chosen host.

Promoter selection is also dependent upon the desired efficiency and level of peptide or protein production. Inducible promoters such as tac are often employed in order to dramatically increase the level of protein expression in E. coli. Overexpression of proteins may be harmful to the host cells. Consequently, host cell growth may be limited. The use of inducible promoter systems allows the host cells to be cultivated to acceptable densities prior to induction of gene expression, thereby facilitating higher product yields.

Various signal sequences may be used according to the invention. A signal sequence which is homologous to the biologically active polypeptide coding sequence may be used. Alternatively, a signal sequence which has been selected or designed for efficient secretion and processing in the expression host may also be used. For example, suitable signal sequence/host cell pairs include the B. subtilis sacB signal sequence for secretion in B. subtilis, and the Saccharomyces cerevisiae α-mating factor or P. pastoris acid phosphatase phoI signal sequences for P. pastoris secretion. The signal sequence may be joined directly through the sequence encoding the signal peptidase cleavage site to the protein coding sequence, or through a short nucleotide bridge consisting of usually fewer than ten codons, where the bridge ensures correct reading frame of the downstream protein sequence.

Elements for enhancing transcription and translation have been identified for eukaryotic protein expression systems. For example, positioning the cauliflower mosaic virus (CaMV) promoter 1000 bp on either side of a heterologous promoter may elevate transcriptional levels by 10- to 400-fold in plant cells. The expression construct should also include the appropriate translational initiation sequences. Modification of the expression construct to include a Kozak consensus sequence for proper translational initiation may increase the level of translation by 10 fold.

A selective marker is often employed, which may be part of the expression construct or separate from it (e.g., carried by the expression vector), so that the marker may integrate at a site different from the gene of interest. Examples include markers that confer resistance to antibiotics (e.g., bla confers resistance to ampicillin for E. coli host cells, nptII confers kanamycin resistance to a wide variety of prokaryotic and eukaryotic cells) or that permit the host to grow on minimal medium (e.g., HIS4 enables P. pastoris or His⁻ S. cerevisiae to grow in the absence of histidine). The selectable marker has its own transcriptional and translational initiation and termination regulatory regions to allow for independent expression of the marker. If antibiotic resistance is employed as a marker, the concentration of the antibiotic for selection will vary depending upon the antibiotic, generally ranging from 10 to 600 μg of the antibiotic/mL of medium.

The expression construct is assembled by employing known recombinant DNA techniques (Sambrook et al., 1989; Ausubel et al., 1999). Restriction enzyme digestion and ligation are the basic steps employed to join two fragments of DNA. The ends of the DNA fragment may require modification prior to ligation, and this may be accomplished by filling in overhangs, deleting terminal portions of the fragment(s) with nucleases (e.g., ExoIII), site directed mutagenesis, or by adding new base pairs by PCR. Polylinkers and adaptors may be employed to facilitate joining of selected fragments. The expression construct is typically assembled in stages employing rounds of restriction, ligation, and transformation of E. coli. Numerous cloning vectors suitable for construction of the expression construct are known in the art (λZAP and pBLUESCRIPT SK-1, Stratagene, La Jolla, Calif., pET, Novagen Inc., Madison, Wis., cited in Ausubel et al., 1999) and the particular choice is not critical to the invention. The selection of cloning vector will be influenced by the gene transfer system selected for introduction of the expression construct into the host cell. At the end of each stage, the resulting construct may be analyzed by restriction, DNA sequence, hybridization and PCR analyses.

The expression construct may be transformed into the host as the cloning vector construct, either linear or circular, or may be removed from the cloning vector and used as is or introduced onto a delivery vector. The delivery vector facilitates the introduction and maintenance of the expression construct in the selected host cell type. The expression construct is introduced into the host cells by any of a number of known gene transfer systems (e.g., natural competence, chemically mediated transformation, protoplast transformation, electroporation, biolistic transformation, transfection, or conjugation) (Ausubel et al., 1999; Sambrook et al., 1989). The gene transfer system selected depends upon the host cells and vector systems used.

For instance, the expression construct can be introduced into S. cerevisiae cells by protoplast transformation or electroporation. Electroporation of S. cerevisiae is readily accomplished, and yields transformation efficiencies comparable to spheroplast transformation.

The present invention further provides a production process for isolating a fusion protein of interest. In the process, a host cell (e.g., a yeast, fungus, insect, bacterial or animal cell), into which has been introduced a nucleic acid encoding the protein of the interest operatively linked to a regulatory sequence, is grown at production scale in a culture medium to stimulate transcription of the nucleotides sequence encoding the fusion protein of interest. Subsequently, the fusion protein of interest is isolated from harvested host cells or from the culture medium. Standard protein purification techniques can be used to isolate the protein of interest from the medium or from the harvested cells. In particular, the purification techniques can be used to express and purify a desired fusion protein on a large-scale (i.e. in at least milligram quantities) from a variety of implementations including roller bottles, spinner flasks, tissue culture plates, bioreactor, or a fermentor.

An expressed protein fusion complex can be isolated and purified by known methods. Typically the culture medium is centrifuged or filtered and then the supernatant is purified by affinity or immunoaffinity chromatography, e.g. Protein-A or Protein-G affinity chromatography or an immunoaffinity protocol comprising use of monoclonal antibodies that bind the expressed fusion complex. The fusion proteins of the present invention can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatography, methods utilizing a difference in hydrophobicity such as reverse-phase high performance liquid chromatography and methods utilizing a difference in isoelectric point, such as isoelectric focusing electrophoresis, metal affinity columns such as Ni-NTA. See generally Sambrook et al. and Ausubel et al. supra for disclosure relating to these methods.

It is preferred that the fusion proteins of the present invention be substantially pure. That is, the fusion proteins have been isolated from cell substituents that naturally accompany it so that the fusion proteins are present preferably in at least 80% or 90% to 95% homogeneity (w/w). Fusion proteins having at least 98 to 99% homogeneity (w/w) are most preferred for many pharmaceutical, clinical and research applications. Once substantially purified the fusion protein should be substantially free of contaminants for therapeutic applications. Once purified partially or to substantial purity, the soluble fusion proteins can be used therapeutically, or in performing in vitro or in vivo assays as disclosed herein. Substantial purity can be determined by a variety of standard techniques such as chromatography and gel electrophoresis.

The present fusion protein complexes are suitable for in vitro or in vivo use with a variety of cells that are cancerous or are infected or that may become infected by one or more diseases.

Human interleukin-15 (hIL-15) is trans-presented to immune effector cells by the human IL-15 receptor α chain (hIL-15Rα) expressed on antigen presenting cells. IL-15Rα binds hIL-15 with high affinity (38 pM) primarily through the extracellular sushi domain (IL-15RαSu). As described herein, the IL-15 and IL-15RαSu domains can be used to generate a soluble complex (e.g., ALT-803) or as a scaffold to construct multi-domain fusion complexes.

IgG domains, particularly the Fc fragment, have been used successfully as dimeric scaffolds for a number of therapeutic molecules including approved biologic drugs. For example, etanercept is a dimer of soluble human p75 tumor necrosis factor-α (TNF-α) receptor (sTNFR) linked to the Fc domain of human IgG1. This dimerization allows etanercept to be up to 1,000 times more potent at inhibiting TNF-α activity than the monomeric sTNFR and provides the fusion with a five-fold longer serum half-life than the monomeric form. As a result, etanercept is effective at neutralization of the pro-inflammatory activity of TNF-α in vivo and improving patient outcomes for a number of different autoimmune indications.

In addition to its dimerization activity, the Fc fragment also provides cytotoxic effector functions through the complement activation and interaction with Fcγ receptors displayed on natural killer (NK) cells, neutrophils, monocyte cells, phagocytes and dendritic cells. In the context of anti-cancer therapeutic antibodies and other antibody domain-Fc fusion proteins, these activities likely play an important role in efficacy observed in animal tumor models and in cancer patients. However these cytotoxic effector responses may not be sufficient in a number of therapeutic applications. Thus, there has been considerable interest in improving and expanding on the effector activity of the Fc domain and developing other means of increasing the activity or recruitment of cytolytic immune responses, including NK cells and T cells at the disease site via immunotherapeutic molecules.

In an effort to develop human-derived immunostimulatory therapeutic, human IL-15 (hIL-15) and IL-15 receptor domains were used. hIL-15 is a member of the small four α-helix bundle family of cytokines that associates with the hIL-15 receptor α-chain (hIL-15Rα) with a high binding affinity (Equilibrium dissociation constant (KD)˜10⁻¹¹M). The resulting complex is then trans-presented to the human IL-2/15 receptor β/common γ chain (hIL-15RβγC) complexes displayed on the surface of T cells and NK cells. This cytokine/receptor interaction results in expansion and activation of effector T cells and NK cells, which play an important role in eradicating virally infected and malignant cells. Normally, hIL-15 and hIL-15Rα are co-produced in dendritic cells to form complexes intracellularly that are subsequently secreted and displayed as heterodimeric molecules on cell surfaces. Some studies suggest that IL-15/IL-15Rα complexes are cleaved from the cell surface and released as a soluble functional form. Thus, the characteristics of hIL-15 and hIL-15Rα interactions suggest that these inter chain binding domains could serve as a human-derived immunostimulatory complex and as a scaffold to make soluble dimeric molecules capable of target-specific binding.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Materials and Methods

### Mice and Bone Marrow Transplant (BMT)

Female C57BL/6 (B6, H-2K^{b}), Balb/c (H-2K^{d}), B6CBAF1 (H-2K^{b/k}), CB6F1 (H-2K^{b/d}) and B6D2F1 (H2K^{b/d}) mice were obtained from the Jackson Laboratory (Bar Harbor, Me.). Mice used in BMT experiments were between 10-12 weeks of age. BMT protocols were approved by the Institutional Animal Care and Use Committee (IACUC) at Thomas Jefferson University.

Bone marrow (BM) cells were removed aseptically from femurs and tibias and T cells were depleted (TCD) by incubation with anti-Thy 1.2 antibody for 30 min at 4° C., followed by incubation with Low-TOX-M rabbit complement (Cedarlane Laboratories, Hornby, Ontario, Canada) for 40 minutes at 37° C., or alternatively via anti-CD5 magnetic bead depletion (Miltenyi, Auburn, Calif.). Typical levels of contaminating T cells after complement depletion ranged from 0.2 to 0.5 percent of all bone marrow leukocytes.

Splenic T cells were obtained by positive selection with anti-CD5 antibodies conjugated to magnetic beads (Miltenyi, Auburn, Calif.). In some cases, CD4⁺ and CD8⁺ T cell populations were separated out individually (Miltenyi, Auburn, Calif.). Cells (5×10⁶ BM cells with or without splenic T cells) were resuspended in Dulbecco Modified Eagle's Medium (DMEM) and transplanted by tail vein infusion (0.25 ml total volume) into lethally irradiated recipients on day 0. On day 0 prior to transplantation, recipients received 11 to 13 Gy total body irradiation (strain dependent) from a ¹³⁷Cs source as a split dose with a 3 hour interval between doses to reduce gastrointestinal toxicity. Mice were housed in sterilized micro-isolator cages and received normal chow and autoclaved hyper-chlorinated drinking water (pH 3.0).

### Cell Lines, Antibodies, and Cytokines

P-815 (H-2d) cell line was obtained from ATCC (Manassas, Va.). A20 (H-2d) murine lymphoma cell line, retrovirally transduced to express a triple fusion protein consisting of Herpes simplex virus thymidine kinase, enhanced green fluorescent protein (eGFP) and firefly luciferase (TGL), was kindly provided by Dr. Marcel van den Brink (Memorial Sloan Kettering Cancer Center, New York, N.Y.). Cells were cultured in RPMI with 10% FBS in atmosphere containing 5% CO₂.

Anti-murine CD16/CD32 FcR block (2.4G2) and all of the following fluorochrome-labeled antibodies against murine antigens were obtained from BD Pharmingen (San Diego, Calif.): H2Kd (SF1-1.1), CD3 (500A2), CD4 (RM4-5), CD8 (53-6.7), CD25 (PC61), CD44 (IM7), CD45R/B220 (RA3-6B2), CD62L (MEL-14), NK1.1 (PK136), TNF-α (MP6-XT22), IFN-γ (XMG1.2), NK2GD, isotype controls; rat IgG2a-κ, rat IgG1-κ hamster, and IgG1-κ.

ALT-803 was generated by Altor BioScience Corporation, Miramar, Fla. ALT-803 was administered intraperitoneally, weekly at 1-5 μg/day.

### Flow Cytometry

Single cell suspension of 10⁶ cells/25 μL was incubated at 4° C. with CD16/CD32 FcR block. Subsequently, cells were incubated at 4° C. with antibodies in a total volume of 50 μl. The stained cells were analyzed on a FACS Calibur flow cytometer (Becton Dickinson, San Jose, Calif.) with CellQuest software or LSRII cytometer (Becton Dickinson, San Jose, Calif.) with FlowJo software (Treestar, San Carlos, Calif.).

### Assessment of Graft-Versus-Host-Disease

The severity of GVHD was assessed with a clinical GVHD scoring system as previously described (Cooke, K. R., et al., Blood, 1996. 88(8): p. 3230-9). Briefly, ear-punched animals in coded cages were individually scored every week using 5 clinical parameters based on a scale from 0 to 2: weight loss, posture, mobility, fur, and skin. A clinical GVHD index was generated by summation of the 5 criteria scores (0-10). Survival was monitored daily. Animals with scores of at least 5 were considered moribund and were sacrificed.

### PMA-Ionomycin Stimulation and Intracellular Staining

Splenocytes were incubated with PMA (20 ng/mL) and ionomycin (1 μM) for 5 hours. Brefeldin A was added at a concentration of 10 μg/mL two hours following the addition of PMA and ionomycin. Cells were first stained with surface antibodies and then fixed and permeabilized with the BD Cytofix/Cytoperm Kit (BD Biosciences, San Diego, Calif.) and subsequently stained with intracellular antibodies.

### CF SE Labeling

Cells were labeled with carboxyfluorescein succinimidyl ester (CFSE) as previously described (Lyons, A. B. and C. R. Parish, Determination of lymphocyte division by flow cytometry. J Immunol Methods, 1994. 171(1): p. 131-7). Briefly, splenocytes were incubated with CFSE at a final concentration of 2.5 μM in PBS at 37° C. for 20 minutes. Cells were then washed three times with PBS before intravenous injection.

### Statistics

All values shown in graphs represent the mean±SEM. Survival data were analyzed using the Mantel-Cox log-rank test. For all other analysis, nonparametric unpaired Mann-Whitney-U test was used.

### Example 2: Effects of ALT-803 on Immune Cells Following HSCT

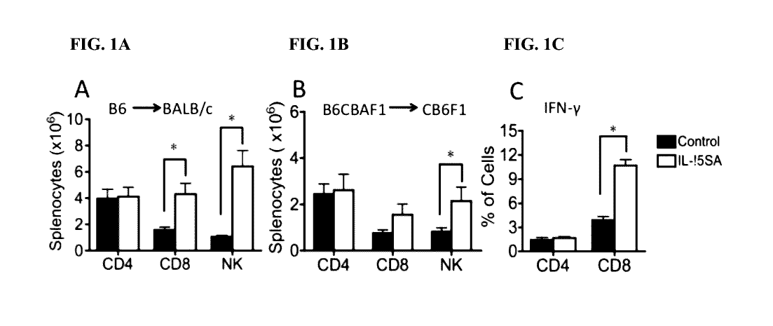

The effects of ALT-803 were first evaluated in T cell depleted models. Lethally irradiated BALB/c recipients were transplanted with T cell depleted (TCD) bone marrow (BM) cells from B6 mice. ALT-803 was administered via intraperitoneal (i.p.) injection in two doses on days 17 and 24 after transplant. Animals were sacrificed on day 28. All recipients had more than 90% engraftment in the spleens and BMs. There was no significant difference in engraftment and cellularity in the spleens and BMs between ALT-803 and control groups. Administration of ALT-803 significantly increased the number of CD8⁺ T and NK cells, whereas there was no change in CD4⁺ T cell numbers (FIG. 1A). Similar activity was observed in the B6CBA→CB6F1 transplant model (FIG. 1B), in which animals were treated with the same dose and schedule. ALT-803 also augmented intracellular IFN-γ secretion by CD8⁺, but not CD4⁺ T cells in this model (FIG. 1C).

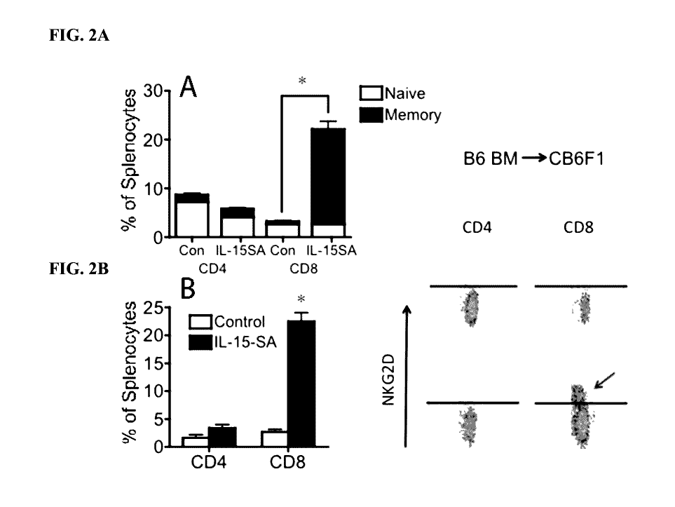

Next, the effects of ALT-803 on CD4⁺ and CD8⁺ naïve (CD44^{low}) and memory (CD44^{high}) T cell populations were evaluated. Again, recipients were treated with 1 μg ALT-803 i.p. on days 28, 35 and 42 after HSCT (B6 CB6F1). ALT-803 administration mostly increased the CD8⁺ memory/effector T cell population, but did not show any activity on both CD4⁺ memory and naïve T cell populations. CD8⁺ naïve T cells also remained unaffected in both ALT-803 treated and untreated groups (FIG. 2A). Other activation markers on the lymphocytes were also evaluated. A 10-fold increase in NKG2D expression was identified on CD8⁺ T cells, suggesting that some CD8⁺ T cells turn into effector/cytotoxic lymphocytes with innate-like phenotype (FIG. 2B) after exposure to ALT-803. These effects of ALT-803 on immune cells after HSCT are similar to previously observed changes in preclinical studies (Wong, H. C., E. K. Jeng, and P. R. Rhode, Oncoimmunology, 2013. 2(11): p. e26442). A significant change in surface CD107a expression was not identified on either CD4⁺ or CD8⁺ T cells, which is a marker of degranulation of the cytolytic perforin/granzyme pathway against tumors.

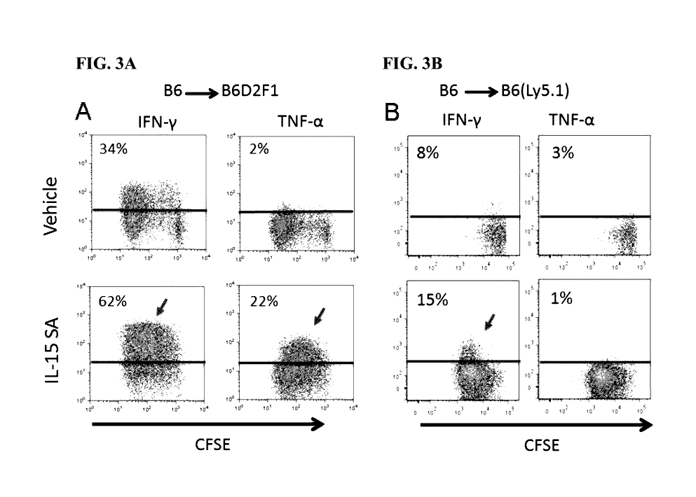

The effects of ALT-803 on the adoptively transferred CFSE-labeled splenocytes from B6 mice into the lethally irradiated B6D2F1 recipient mice were examined. ALT-803 treatment specifically promoted proliferation of slow-proliferating CD8⁺ T cells in conjunction with robust IFN-γ and TNF-α secretion, in allogeneic recipients of CFSE labeled-T-cell infusion. However, there was no effect of ALT-803 on CD4⁺ T cell proliferation (FIG. 3A and FIG. 3B). A significant increase in TNF-α secretion by CD8⁺ T cells following IL-15 administration was not identified in previous experiments (Sauter, C. T., et al., Bone Marrow Transplant, 2013. 48(9): p. 1237-42), suggesting that ALT-803 is more potent than native IL-15 for inducing cytokine secretion in CD8⁺ T cells in vivo. Next, ALT-803 activity was evaluated in syngeneic recipients of CFSE labeled T-cell infusion. Again, it was identified that ALT-803 increased proliferation and IFN-γ secretion in adoptively transferred CD8⁺ T cells, but did not increase their TNF-α secretion (FIG. 3B). These results further suggest that additional stimulatory signals, such as TCR-MHC engagement in the allogeneic, rather than the syngeneic adoptive T cell transfer setting, are potentially necessary to induce TNF-α secretion by ALT-803 stimulation.

### Example 3: Antitumor Activity of ALT-803 in Murine Tumor Models

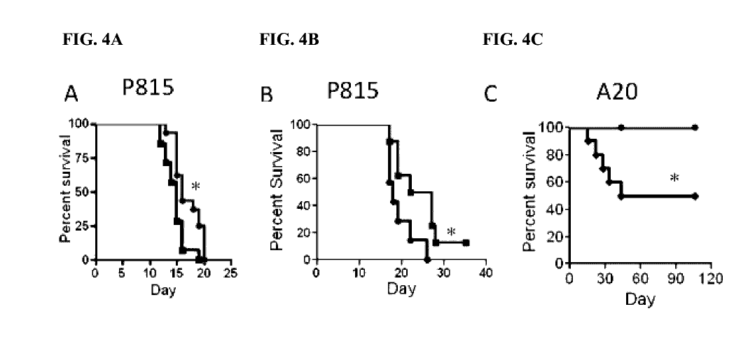

Next, the anti-tumor activity of ALT-803 was examined in two different tumor models—murine mastocytoma (P815) and murine B cell lymphoma (A20). First, anti-tumor activity of ALT-803 was evaluated in the P815 model, without T cell administration. Significant graft-versus-tumor (GVT) activity was not detected in recipients of P815 in parent-F1 model when ALT-803 was administered without the T cell infusion. When a small amount of T cells was infused into the B6→B6D2F1 model, it was identified that ALT-803 administration significantly enhanced anti-tumor activity against P815 tumor cells with two different T cell doses; 5×10⁴ and 1×10⁵ cells, respectively (FIGS. 4A and 4B). Signs of GVHD were not observed in these experiments. All animals died from tumor development with hind leg paralysis or presence of tumor metastasis in the autopsy. Therefore, ALT-803 administered with T cell infusion in the P815 model provided significant survival benefit to the tumor-bearing mice as compared to control.

In the A20 murine lymphoma model, anti-lymphoma activity against A20 cells was evaluated in recipients of allogeneic HSCT, with or without T cell infusion. A20 cells were kindly provided by Dr. van den Brink's laboratory (Memorial Sloan-Kettering Cancer Center, New York, N.Y.), expressing triple gene construct with luciferase activity that allowed for the detection of tumor growth with bioluminescence imaging (BLI). First, an A20 murine tumor model was developed. Infusion of 1×10⁵ donor T cells provided a significant anti-tumor activity and survival benefit in the B6CBAF1→CB6F1 (MHC-mismatched) model. Thus, CB6F1 mice were lethally irradiated and B6CBAF1 BM cells along with 1×10⁵ T cells were transplanted. All animals received A20 tumor cells on the same day as BM transplant. All animals in the ALT-803-treated group survived, which is statistically significant compared to the control group (FIG. 4C).

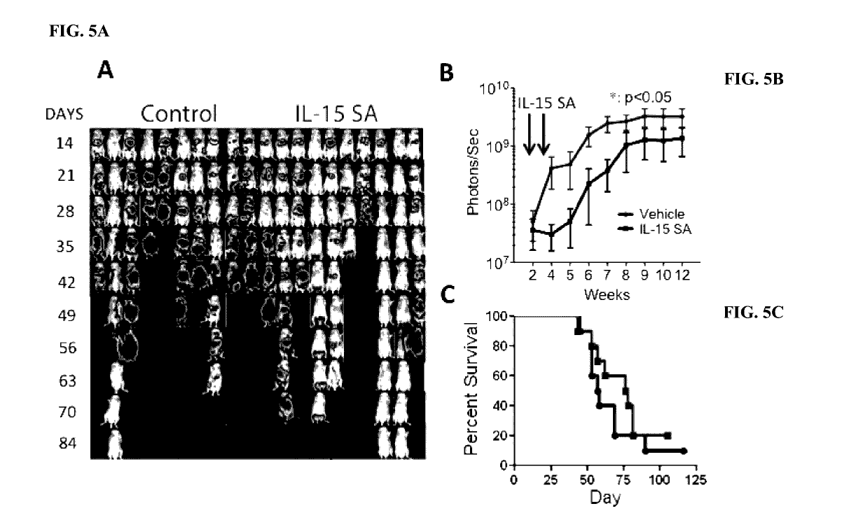

Next, anti-lymphoma/leukemia activity of ALT-803 was explored in allogeneic HSCT recipients without any T cell infusion in the A20 model. Same ALT-803 dose and administration route were used as previously described. Tumor growth was determined by intensity of photon measurements using IVIS bioluminescence system (FIG. 5A and FIG. 5B). Although it was observed that two administrations of ALT-803 could provide a delay of A20 cell growth in vivo, delayed tumor growth by ALT-803 administration did not result in a survival difference between the ALT-803 group and the control group (FIG. 5C). ALT-803 is still successful in generating anti-tumor activity against A20 lymphoma cells without a T cell infusion.

Taken together, the results of these experiments indicate that ALT-803 significantly increases the anti-lymphoma/leukemia activity in murine HSCT by effectively promoting the effector/memory CD8⁺ T and NK cell expansion and potently enhancing their effector functions.

### Example 4: ALT-803 Enhances Anti-Tumor Activity with Donor Leukocyte Infusion (DLI)

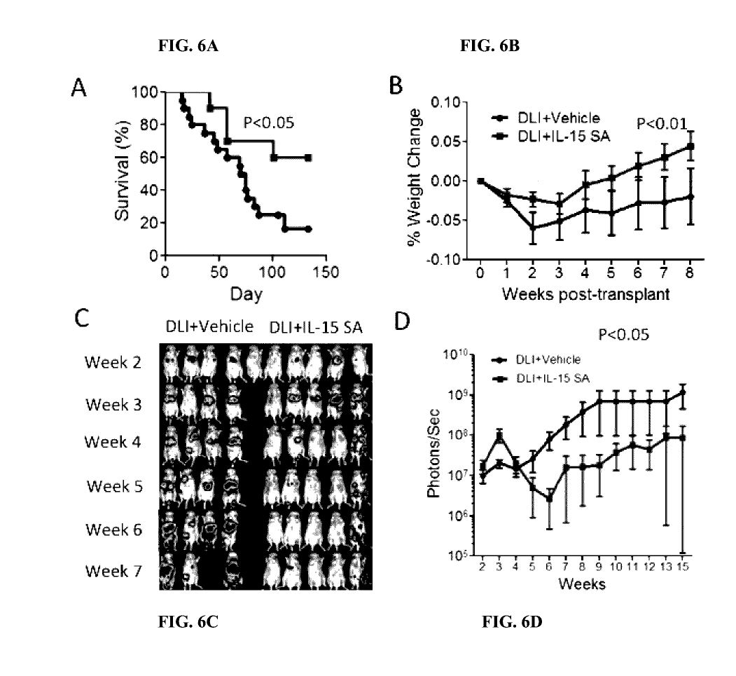

DLI has been developed as a strategy for management of relapse by increasing GVT effects after allogeneic HSCT (Kolb, H. J., et al., Blood, 1990. 76(12): p. 2462-5). DLI is used in nearly all malignant hematological diseases for which allogeneic HSCT is performed. However, the response to DLI varies with respect to the methods of cell collection, timing, cell dose infused, and even cell sub-type used (reviewed in (Tomblyn, M. and H. M. Lazarus, Bone marrow transplantation, 2008. 42(9): p. 569-79). It is conceivable that the enhancement of the efficacy of DLI would improve the outcome the patients who relapse after allogeneic HSCT. Therefore, it was examined whether ALT-803 can be used to enhance the efficacy of DLI in animal models. To achieve this, a DLI model was developed with recipients of allogeneic HSCT. Lethally irradiated CB6F1 recipients were transplanted with T cell-depleted B6 BM cells. A20 murine lymphoma cells were infused at the day of transplant. Purified T cells were infused after tumor growth in recipients of allogeneic HSCT. A moderate dose of T cell infusion (2.5×10⁵ cells) provided GVL activity after tumor development in recipients of allogeneic HSCT. The ALT-803-treated group exhibited a better survival and less weight loss after transplant compared to the untreated group (FIG. 6A and FIG. 6B). Also, tumor growth was evaluated in all animals with BLI. ALT-803 administration resulted in significantly decreased photon intensity by BLI, which suggests ALT-803 was able to inhibit tumor growth (FIG. 6C and FIG. 6D). An increase in GVHD score and weight loss in ALT-803-treated group compared to the control group was not observed (FIG. 6B), suggesting that ALT-803 did not promote GVHD in this murine HSCT model. Thus, ALT-803 administration after allogeneic HSCT enhances GVL/lymphoma activity without aggravating GVHD in murine models.

### Example 5: ALT-803 Significantly Enhances Graft-Versus-Tumor Activity after Allogeneic Hematopoietic Stem Cell Transplantation

Interleukin-15 (IL-15) is a pleiotropic cytokine, which plays various roles in the innate and adaptive immune system, including the development, activation, homing and survival of immune effector cells. IL-15 has been previously shown to increase CD8+ T and NK cells number and function in normal mice and recipients of stem cell transplantation. However, obstacles remain in using IL-15 therapeutically, specifically its low potency and short in vivo half-life. To overcome this, anIL-15 mutant (IL-15N72D; J. Immunol, 2009; 183:3598) has been developed, with increased biological activity. Co-expressing IL-15N72D, in conjunction with IL-15RαSu/Fc produced a biologically active and highly potent IL-15 superagonist complex (IL-155A, also known as ALT-803, Cytokine, 2011; 56:804). The effects of ALT-803 on immune reconstitution and graft-versus-tumor (GVT) activity were evaluated in recipients of allogeneic hematopoietic stem cell transplantation (HSCT). Lethally irradiated BALB/c recipients were transplanted with T-cell depleted (TCD) bone marrow (BM) cells from B6 mice. ALT-803 was administered via IP injection in two doses on days 17 and 24 after transplant. Animals were sacrificed at day 28. Administration of IL-15 significantly increased the numbers of CD8+ T cells and NK cells. ALT-803 also augmented interferon-γ secretion from CD8+ T cells. Similar activity was observed in B6CBA→CB6F1 transplant model. ALT-803 upregulates NKG2D and CD107a expression on CD8+ T cells. ALT-803 administration also specifically increased slow-proliferative CD8+ T-cell proliferation in conjunction with robust IFN-γ and TNF-α secretion in CD8+ T cells in recipients of CFSE (carboxyfluorescein succinimidyl ester) labeled-T-cell infusion, whereas there was no effect on CD4+ T-cell proliferation. Next, the anti-tumor activity of ALT-803 was examined in three different tumor models; murine mastocytoma (P815), murine B cell lymphoma (A20) and murine renal cell carcinoma (Renca). It was determined that ALT-803 administration enhanced GVT activity against P815 and A20 in recipients of allogeneic HSCT though this activity required a low-dose T cell infusion with HSCT. Augmented GVT activity against Renca after ALT-803 administration in recipients of allogeneic HSCT did not require T cell infusion.

In conclusion, ALT-803 is a very potent cytokine complex for enhancing CD8+ and NK T cell reconstitution and function after HSCT, which would be a candidate for post-transplant immunotherapy.

### Example 6: ALT-803 Combined with Donor Lymphocyte Infusion (DLI) Significantly Enhances Graft-Versus-Tumor Activity after Allogeneic Hematopoietic Stem Cell Transplantation

Donor lymphocyte infusion (DLI) has been successfully used clinically to augment the graft-versus-tumor (GVT) effect following hematopoietic stem cell transplantation (HSCT) in relapsed patients. However, improvements can still be made in enhancing anti-tumor activity, reducing graft-versus-host disease (GVHD) and decreasing complications from opportunistic infections. The results described herein present clear evidence of increased tumor clearance via cytokine therapy in combination with DLI as a way to “boost” the infused cells function.

Interleukin-15 (IL-15) is a potent cytokine that increases CD8+ T and NK cells number and function in normal mice and recipients of stem cell transplantation. Despite this, prior to the invention described herein, there were obstacles for the use of IL-15 therapeutically, specifically its low potency and short in vivo half-life. To overcome this, ALT-803 has been developed with a longer half-life and increased potency.

As described herein, administration of ALT-803 to recipients of CFSE labeled T cells increases proliferation of CD8+ T cells and IFN-γ and TNF-α secretion from CD8+ T cells. A murine DLI model was developed by titrating the dose of infused T cells in a parent-F1 model, and then combined ALT-803 administration with DLI in murine recipients of allogeneic HSCT. In this model, lethally irradiated CB6F1 (H2K^{b/d}) mice were transplanted with T-cell depleted bone marrow cells from C57BL6 mice (H2K^{b}). All recipients of HSCT were also co-injected A20 B-cell lymphoma cells transfected with a luciferase-producing gene, which allows bioluminescent imaging and tracking of tumor progress in vivo. Mice receiving DLI (2.5×10⁵ T cells) with ALT-803 injections given at 1 μg/mouse on days 17 and 24 post-BMT show less tumor burden and increased overall survival (p=0.04) and decreased tumor growth (p=0.02). The ALT-803 treated group had a significantly less weight loss than the control group (p=0.007). No GVHD symptoms were noted via weekly clinical scoring, highlighting both the efficacy and overall safety of the ALT-803 therapy. Furthermore, T-cell exhaustion markers were evaluated on CD8+ T cells in surviving mice. Increased programmed death-1 (PD-1) expression was found on T cells even when the tumor burden is cleared. Treatment with ALT-803 reduced PD-1 expression on donor CD8+ T-cells in mice surviving more than 120 days post-transplant.

In conclusion, ALT-803 enhanced CD8+ T cell function by increasing cytokine secretion and proliferation of T cells whereas could also prevent T cell exhaustion. ALT-803 is a long-waited lymphoid growth factor and is useful in combination with DLI for the treatment of recurrent disease after HSCT.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for treating a neoplasia in a subject, the method comprising:
administering to said subject an effective amount of an adoptive cell therapy and an effective amount of a pharmaceutical composition comprising an IL-15:IL-15Rα complex,
thereby treating the neoplasia.

2. The method of claim 1, wherein the IL-15/IL15Rα complex is an IL-15N72D:IL-15RαSu/Fc complex (ALT-803), wherein said ALT-803 comprises a dimeric IL-15RαSu/Fc and two IL-15N72D molecules.

3. The method of claim 2, wherein the IL-15N72D molecule comprises SEQ ID NO: 3.

4. The method of claim 2, wherein the IL-15RαSu/Fc comprises SEQ ID NO: 6.

5. The method of claim 1, wherein the neoplasia is selected from the group consisting of hematological cancer, chronic myelogenous leukemia, acute myelogenous leukemia, acute lymphoblastic leukemia, myelodysplasia, multiple myeloma, mantle cell lymphoma, B cell non-Hodgkin lymphoma, Hodgkin's lymphoma, chronic lymphocytic leukemia, B-cell neoplasms, B-cell lymphoma, leukemia, cutaneous T-cell lymphoma, T-cell lymphoma, a solid tumor, urothelial/bladder carcinoma, melanoma, lung cancer, renal cell carcinoma, breast cancer, gastric and esophageal cancer, head and neck cancer, prostate cancer, colorectal cancer, ovarian cancer, non-small cell lung carcinoma, sarcoma, mastocytoma and adenocarcinoma.

6. The method of claim 2, wherein the effective amount of said ALT-803 is administered once or twice per week.

7. The method of claim 2, wherein the effective amount of said ALT-803 is administered daily.

8. The method of claim 6, wherein the effective amount of said ALT-803 is between 0.1 μg/kg and 100 mg/kg.

9. The method of claim 1, wherein said pharmaceutical composition is administered systemically, intravenously, subcutaneous, intramuscularly, intravesically, or by instillation.

10. The method of claim 1, wherein said adoptive cell therapy comprises hematopoietic stem cell transplantation, donor leukocyte infusion, or adoptive transfer of T cells or NK cells.

11. The method of claim 1, wherein said adoptive cell therapy comprises transfer of allogeneic, autologous, syngeneic, related, unrelated, MHC-matched, MHC-mismatched or haploidentical cells.

12. The method of claim 10, wherein said T cells or NK cells are engineered to express a exogenous suicide gene, chimeric antigen receptor, or T cell receptor.

13. The method of claim 1, wherein said ALT-803 stimulates proliferation or activation of adoptively transferred cells.

14. The method of claim 13, wherein said ALT-803 increases the number of adoptively transferred CD8⁺ T cells or NK cells in said subject.

15. The method of claim 13, wherein said ALT-803 increases expression of IFN-γ, TNF-α, NKG2D or CD107a in said adoptively transferred cells.

16. The method of claim 1, wherein said administration increases graft-verse-tumor activity.

17. The method of claim 1, wherein said administration does not increase graft-verse-host disease.

18. The method of claim 1, wherein said administration results in a decreased number of tumor cells.

19. The method of claim 1, wherein said administration results in a decrease in progression or a decrease in relapse of the neoplasia.

20. The method of claim 1, wherein said administration results in prolonged survival of said subject compared to an untreated subject.

21. The method of claim 1, wherein said subject is a human.

22. The method of claim 1, wherein said subject has a neoplasia that has relapsed from or is refractory to therapy administered previously.

23. The method of claim 1, wherein said adoptive cell therapy and said ALT-803 are administered simultaneously or sequentially.

24. A method for treating a subject with a neoplasia that has relapsed from previous therapy, the method comprising:
administering to said subject an effective amount of an donor lymphocyte infusion therapy and an effective amount of ALT-803,
thereby treating the neoplasia that has relapsed from previous therapy.

25. The method of claim 24, wherein said method does not induce graft-verse-host disease.

26. The method of claim 24, further comprising identifying a subject with a neoplasia who has relapsed from previously-administered therapy.

27. A kit for the treatment of a neoplasia, the kit comprising an effective amount of ALT-803, an adoptive cell therapy, and directions for the use of the kit for the treatment of a neoplasia.

28. The kit of claim 27, wherein said adoptive cell therapy comprises hematopoietic stem cells, donor leukocytes, T cells, or NK cells.


SEQUENCE LISTING (6 sequences)

SEQ ID NO: 1 - DNA, 405 bp, Artificial Sequence
  Description of Artificial Sequence Synthetic polynucleotide
atggagacagacacactcctgttatgggtactgctgctctgggttccaggttccaccggtaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacgacagtttgtcttctaatgggaatgtaacagaatctggatgcaaagaatgtgaggaactggaggaaaaaaatattaaagaatttttgcagagttttgtacatattgtccaaatgttcatcaacacttcttaa

SEQ ID NO: 2 - protein, 134 aa, Artificial Sequence
  Description of Artificial Sequence Synthetic polypeptide
METDTLLLWVLLLWVPGSTGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 3 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 4 - DNA, 951 bp, Artificial Sequence
  Description of Artificial Sequence Synthetic polynucleotide
atggacagacttacttcttcattcctgctcctgattgtccctgcgtacgtcttgtccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgtattagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 5 - protein, 316 aa, Artificial Sequence
  Description of Artificial Sequence Synthetic polypeptide
MDRLTSSFLLLIVPAYVLSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 297 aa, Artificial Sequence
  Description of Artificial Sequence Synthetic polypeptide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK